(19) United States
(12) Patent Application Publication
(10) Pub. No.: US 2011/0245750 A1
(43) Pub. Date: Oct. 6, 2011

(54) METHOD AND APPARATUS FOR ACOUSTICALLY ENHANCED REMOVAL OF BUBBLES FROM A FLUID

(76) Inventors: John E Lynch, Williamsburg, VA (US); Christopher S. Domack, Carrollton, VA (US); Bill B. Hefner, JR., Williamsburg, VA (US)

(21) Appl. No.: 13/063,547

(22) PCT Filed: Sep. 8, 2009
(86) PCT No.: PCT/US09/56154

§ 371 Date: Jun. 13, 2011

Related U.S. Application Data
(60) Provisional application No. 61/096,080, filed on Sep. 11, 2008.
(60) Provisional application No. 61/184,190, filed on Jun. 4, 2009.

(51) Int. Cl. A61M 1/36; B01D 19/00

(52) U.S. Cl. 604/5.01; 96/175; 95/260

(57) ABSTRACT

A vessel for removing bubbles from a fluid is provided. The vessel includes a fluid inlet port for receiving the fluid and a bubble outlet for removing bubbles in the fluid from the vessel. One or more ultrasonic transducers transmit one or more ultrasonic beams through the received fluid to move bubbles in the fluid towards the bubble outlet. A fluid outlet port outputs the fluid insonified by the one or more ultrasonic beams. A conduit structure conveys the one or more ultrasonic beams through the vessel in a first direction towards the air outlet. An interface prevents reflection of one or more ultrasonic beams in a direction generally opposite the first direction.

### RELATED APPLICATIONS

This application claims priority from U.S. Provisional Patent Application Ser. Nos. 61/096,080 and 61/184,190, filed respectively on Sep. 11, 2008 and Jun. 4, 2009, the disclosures of which are incorporated herein by reference in their entirety.

### TECHNICAL FIELD

The technology relates to removing of bubbles of gas from a fluid. One non-limiting example application is to the removal of gaseous emboli from blood circulated in an extracorporeal blood circuit, such as in a heart-lung machine or in a dialysis machine.

### BACKGROUND

An embolus is a structure that travels through the bloodstream, lodges in a blood vessel and blocks it. Examples of emboli are a detached blood clot, a clump of bacteria, foreign material, and air bubbles. In surgical operations, heart surgery in particular, there is a relationship between increased number of emboli present in blood delivered to the brain, i.e., the embolic load delivered to the brain, and neurocognitive deficits. As a result, arterial line filters may be employed in an extracorporeal blood (CPB) circuit to filter out emboli from the blood circulating in the circuit. Unfortunately, arterial line filters include pores large enough, e.g., 28 to 40·10⁻⁶ m (28 to 40 μm), to allow smaller emboli to pass through, and larger air and fat emboli also pass through and enter the circulation downstream to the filter whenever their load is high. Significantly, microbubbles that pass through the arterial line filter join together and become large bubbles potentially causing harm to the patient. This problem is particularly severe in low-prime bypass circuits, so that despite their advantages (e.g., lower prime volume results in higher hematocrit values, less systemic inflammation, less platelet activation, and better oxygen delivery to the patient), low-prime bypass circuits do not purge venous air from the system as well as traditional bypass circuits.

So there is a need for a better method for removing gaseous emboli from the bypass circuit prior to returning blood to the patient. The inventors considered various ways to remove gaseous emboli from the blood. One way is to increase the amount of fluid in a bubble removal vessel in the CBP circuit, for example, by increasing the cross-sectional area of a bubble removal vessel. This increased cross-section effectively slows down blood flow in the CBP bubble removal vessel which makes bubbles easier to trap and remove. A wider cross-sectional area also creates a smaller pressure drop from the inlet to the outlet so that the buoyant force of the bubble may be used to separate air bubbles from blood. Similarly, CBP bubble removal components may be made taller to give the bubbles more time to overcome the flow velocity and rise into a gas purge outlet in the vessel. However, there is a limit to the size of the vessel that may be used during bypass—as larger vessels require greater dilution of blood with a priming solution and increased use of transfused blood. It would be desirable to remove microbubbles from a bypass circuit while reducing the size of the circuit, thus leading to reduced dependence on transfused blood during cardiopulmonary bypass surgery.

Another way to remove air from blood is by causing the blood to move in a swirling flow so that air bubbles are pulled to the center of the swirl as in a centrifuge. Alternatively, the pressure within a CBP bubble removal component may be controlled to discourage formation of gas bubbles. Both techniques can be effective in removing larger bubbles, but do not remove microbubbles which are more difficult to remove from flowing blood due to their reduced buoyant force.

Ultrasound waves, which have acoustic radiation force, can used to actively remove bubbles. An ultrasonic wave carries momentum that is transferred to a particle, e.g., an air bubble, upon reflection or absorption of the sound wave.

What is needed is technology that can remove both large bubbles and microbubbles. The technology in the parent U.S. patent application Ser. No. 12/129,985 does that and includes a vessel with three chambers: a fluid inlet chamber, a fluid outlet chamber, and an ultrasonic standoff region. A barrier between the fluid inlet chamber and the fluid outlet chamber prevented fluid from passing between the two chambers without passing through an ultrasonic beam whose beam width matches the opening between the two chambers. That design works well to remove both microbubbles and larger air bubbles in fluid flowing at rates of two liters per minute. But at higher flow rates in some applications, e.g., bubble removal from blood, the opening between the two chambers must be larger in order to slow the flow velocity down. Otherwise, the ultrasound beam must have very high power densities in order to provide sufficient force to push the bubbles against the flow of the blood fluid. Such high power densities can damage cells within the blood, and transducers operating at these power levels are prone to failure. An alternative is to widen the opening between the fluid inlet and outlet chambers so that the flow velocity is lower for a given volume flow rate. But this opening needs to be fairly large which means that the ultrasonic beam needs a fairly large diameter, and the ultrasonic beam would need a high power density within the opening. For example, an opening of approximately three inches in diameter would need an ultrasonic beam power density within the opening of approximately 10 W/cm². Large-area transducers required to generate beams of this diameter and power are difficult to produce and are also prone to failure due to the multiple vibrational modes generated in over the larger surface area.

### SUMMARY

The ultrasonic bubble removal technology described here removes bubbles including very small microbubbles from a fluid. In addition to ultrasonic bubble removal, additional bubble removal mechanisms are used to enhance the reliability of bubble removal. These additional bubble removal mechanisms also ensure that ultrasonic power levels can be kept below established safety guidelines in sensitive applications like CBP gaseous emboli removal.

A first, non-limiting example embodiment provides a vessel for removing bubbles from a fluid. The vessel includes a fluid inlet port for receiving the fluid, and a bubble outlet port for removing bubbles in the fluid from the vessel. An ultrasonic transducer is mounted in the vessel and transmits an ultrasonic beam through the received fluid to move bubbles in the fluid towards the bubble outlet port. A fluid outlet port outputs the fluid insonified by the ultrasonic beam. An ultrasonic reflector mounted near the bubble outlet port reflects the ultrasonic beam away from the fluid outlet port to reduce or prevent reflection of the ultrasonic beam off an interior surface in the vessel directed towards the fluid outlet port. Preferably, the reflector is mounted to reflect the ultrasonic beam away from the fluid outlet port but also in way that increases the amount of acoustic radiation force directed towards the bubble outlet port.

The vessel may include a barrier having a first barrier portion that separates the fluid inlet port and the fluid outlet port. An opening in the first barrier portion permits the ultrasonic beam to radiate fluid received from the fluid inlet port and allows the received fluid from the fluid inlet port to reach the fluid outlet port. In a non-limiting preferred embodiment, the opening is sized to at least substantially match a width of the ultrasonic beam, and the reflector is positioned to reflect the ultrasonic beam away from the opening. The barrier includes a second barrier portion at a sufficient angle to the first barrier portion to create the opening, the second barrier portion extending past the fluid outlet port. In a non-limiting preferred embodiment, the first barrier portion is substantially perpendicular to a sidewall of the vessel, and the first barrier portion and the second barrier portion are substantially perpendicular. The opening may be circular and the second barrier portion cylindrically-shaped. In an alternative example configuration, the second barrier portion includes concentric cylindrically-shaped surfaces.

The vessel preferably includes an acoustically transparent material separating the ultrasonic transducer from the fluid inlet port and the fluid outlet port. A cooling fluid inlet receives cooling fluid that removes heat from the vessel caused by the ultrasonic transducer, and a cooling fluid outlet removes the cooling fluid from the vessel. Alternatively, the fluid may be cooled using radiator fins or similar heat removal structure with or without cooling fluid. The acoustically transparent material prevents the cooling fluid from contacting the received fluid. In one example configuration, the acoustically transparent material is shaped to adjust the ultrasound beam so that a profile of the ultrasound beam approximates the dimensions of the opening in the barrier. The acoustically transparent material defines an ultrasonic standoff region in the vessel between the acoustically transparent material and the ultrasonic transducer. In a non-limiting example additional aspect of the first embodiment, the length of the ultrasonic standoff region substantially matches a near-field/far-field transition of the ultrasonic beam where the ultrasonic wave is at a maximum amplitude.

Different non-limiting configurations of the vessel are described. For example, the ultrasound beam, the opening in the barrier, and the acoustic reflector may be substantially aligned along a same axis. The bubble outlet port may be substantially aligned along the same axis, or it may be offset from and not aligned with the same axis. The ultrasonic transducer may be shaped to focus the energy of the ultrasonic beam through the opening. If the vessel is cylindrically-shaped, the fluid inlet port and the fluid outlet port are preferably oriented substantially tangential to a cylindrical surface of the vessel to produce a swirling flow of the received fluid in the vessel that forces bubbles to the center of the vessel in line with the opening and coalesces smaller ones of the bubbles into larger bubbles. The bubble outlet is preferably located at or near a highest point of the vessel when the vessel is mounted for operation.

In a non-limiting example additional aspect of the first embodiment, the vessel includes a porous mesh positioned in a direction that is substantially parallel to the first barrier portion and covers the opening. The porous mesh mechanically traps bubbles larger than a pore size of the porous mesh, and the ultrasonic beam forces the bubbles towards the bubble outlet port. Alternatively, porous mesh may be positioned in a direction having a substantial angle with the first barrier portion between the fluid inlet port and the opening and between the opening and the fluid outlet port. The angled mesh provides greater surface area for trapping bubbles and reduces the possibility of clogging the mesh with particles that could obstruct flow.

One example advantageous application of the first embodiment is a system for removing gaseous emboli from blood. The system includes a blood circuit receiving blood from a patient. A pump coupled to the blood circuit pumps the blood through the blood circuit. A vessel coupled to the blood circuit removes gaseous emboli from blood. The vessel includes a blood inlet port for receiving the blood, and an emboli outlet port for removing gaseous emboli in the blood from the vessel. An ultrasonic transducer mounted in the vessel that transmits an ultrasonic beam through the received fluid to move gaseous emboli in the fluid towards the gaseous emboli outlet port. A blood outlet port of the vessel outputs the blood insonified by the ultrasonic beam. An ultrasonic reflector mounted near the gaseous emboli outlet port reflects the ultrasonic beam away from the blood outlet port to reduce or prevent reflection of the ultrasonic beam off an interior surface in the vessel directed towards the blood outlet port. The reflector is mounted to reflect the ultrasonic beam away from the gaseous emboli outlet port and to increase an amount of acoustic radiation force directed upwards towards the gaseous emboli outlet port. The system includes a controller for controlling the ultrasonic transducer and the pump.

The blood circuit preferably includes a sensor for sensing gaseous emboli in the blood entering the vessel and providing sensor information to the controller for use by the controller in controlling operation of the ultrasonic transducer. Another sensor for sensing gaseous emboli in the blood exiting the vessel may also be used to detect when gaseous emboli still remains in the blood.

The vessel may be provided in variety of locations in the blood circuit. For example, the vessel may be provided in one or more of the following blood circuit components: a venous reservoir, an arterial line filter, or a bubble trap.

A method for debubbling a liquid in accordance with the first embodiment is also described. The liquid is introduced to a vessel through a fluid inlet and flows through the vessel, preferably in a spiral path, toward a first outlet. An ultrasonic transducer within the vessel transmits an ultrasonic beam along a longitudinal axis the vessel toward the spiral path and toward a second outlet. The ultrasonic beam reflects within the vessel away from the blood outlet port to reduce or prevent reflection of the ultrasonic beam off an interior surface in the vessel directed towards the first outlet. The ultrasonic beam is also reflected away from the second outlet to increase an amount of acoustic radiation force directed upwards towards the second outlet. A stream of insonified liquid is withdrawn through the first outlet, and a stream of liquid containing entrained air bubbles is withdrawn through the second outlet.

A second, non-limiting example embodiment also provides a vessel for removing bubbles from a fluid. The vessel includes a fluid inlet port for receiving the fluid, and an air outlet port for removing air in the fluid from the vessel. One or more ultrasonic transducers transmit one or more ultrasonic beams in a first direction through the received fluid to move bubbles in the fluid towards the air outlet port. A fluid outlet port outputs the fluid insonified by the ultrasonic beam. A conduit structure directs the ultrasonic beam(s) in a first direction. A cross section of the conduit structure preferably substantially matches the cross section of the one or more ultrasonic beam(s). An interface prevents reflection of the one or more ultrasonic beam(s) in an opposite direction from the first direction.

In a first example implementation of the second embodiment, multiple ultrasonic beams are transmitted via corresponding multiple conduits, where the number of conduits preferably matches the number of ultrasonic beams. For example, the conduits might be tubes so that if there are 12 ultrasonic beams, there would be 12 tubes, each tube directing its ultrasonic beam in the first direction.

In non-limiting example implementation of the second embodiment the acoustic reflector is eliminated, and the top portion of the vessel is made of a material whose acoustic impedance, such as an epoxy resin or a plastic, closely matches that of the bubbly fluid. The material may also include an acoustic absorber, such as tungsten power, embedded within to absorb the acoustic energy of the ultrasound wave before it reflects back into the vessel. The material may also be angled to direct reflected energy substantially away from the connecting tubes so that the ultrasound beam energy is dissipated over multiple passes through the interface.

In another non-limiting example implementation of the second embodiment, the vessel is not completely filled with a bubbly fluid, but instead there is a significant fluid/air interface such as in a reservoir. In this case, the radiation force of the sound wave produces a phenomenon known as “acoustic streaming” that results in a small arc in the fluid/air interface. This acoustic streaming arc alters the geometry of the fluid/air interface and dissipates much of the energy incident the sound wave so that little ultrasound energy is reflected back into the vessel.

### BRIEF DESCRIPTION OF THE DRAWINGS

FIG. 1(a) is a non-limiting example of an extracorporeal blood (CPB) circuit n which gaseous emboli are removed;

FIG. 1(b) is another non-limiting example CPB circuit in which gaseous emboli are removed;

FIG. 1(c) is another non-limiting example CPB circuit in which gaseous emboli are removed;

FIG. 2 is a front, perspective view of an ultrasound-assisted debubbling apparatus in accordance with a first non-limiting example embodiment;

FIG. 3 is a cross-sectional view of the ultrasound-assisted debubbling apparatus in FIG. 2;

FIG. 4 a three dimensional, perspective, cross-sectional view of the ultrasound-assisted debubbling apparatus of FIG. 2;

FIG. 5 is a top view of the ultrasound-assisted debubbling apparatus of FIG. 2;

FIG. 6 is a partial cross-sectional view of the ultrasound-assisted debubbling apparatus of FIG. 2 showing ultrasonic beam reflections;

FIG. 7 is a cross-section of the ultrasound-assisted debubbling apparatus of FIG. 2 showing a non-limiting example embodiment with a curved ultrasonic transducer for shaping the ultrasonic beam;

FIG. 8 is a cross-sectional view showing an alternative example embodiment of the debubbling apparatus in FIG. 2 showing a non-limiting example embodiment with a differently-shaped acoustic window separating an ultrasonic stand-off region from fluid regions in the vessel;

FIG. 9 is a cross-sectional view of the debubbling apparatus in FIG. 2 showing a non-limiting example embodiment with the barrier structure between the fluid inlet and fluid outlet ports having a concentrically-shaped portion;

FIG. 10 is a partial cross-sectional view of the debubbling apparatus in FIG. 2 showing an alternative example embodiment where the bubble outlet port is off-center;

FIG. 11 is a side view of an alternative example embodiment for delivering fluid to the debubbling apparatus shown in FIG. 2;

FIGS. 12(a) and 12(b) are cross-sectional views of alternative example embodiments of the debubbling apparatus in FIG. 2 employing one or more porous meshes to filter bubbles;

FIG. 13 is a side cross-sectional view of an ultrasound-assisted debubbling apparatus in accordance with a first implementation of a second non-limiting example embodiment;

FIG. 14 is a side view of the ultrasound-assisted debubbling apparatus shown in FIG. 13;

FIG. 15 is a top view of the ultrasound-assisted debubbling apparatus shown in FIG. 13;

FIG. 16 is a bottom view of the ultrasound-assisted debubbling apparatus shown in FIG. 13;

FIG. 17 shows a representative ultrasound beam tracing for a side cross-sectional view of the ultrasound-assisted debubbling apparatus shown in FIG. 13;

FIG. 18 shows an ultrasound beam profile for a side cross-sectional view of the ultrasound-assisted debubbling apparatus shown in FIG. 13;

FIGS. 19 and 20 are screen shots showing test results of bubble tracks in blood before and after debubbling in the ultrasound-assisted debubbling apparatus shown in FIG. 13;

FIG. 21 is a debubbling model with ultrasound field directed against the direction of fluid flow;

FIG. 22 is a debubbling model with ultrasound field directed perpendicular to the direction of fluid flow;

FIGS. 23(a) and 23(b) show a front face and back face of an example segmented, large-area transducer for use in an the ultrasound-assisted debubbling apparatus;

FIG. 24 is a side cross-sectional view of an open-configuration ultrasound-assisted debubbling apparatus in accordance with a second implementation of the second non-limiting example embodiment using a large-area ultrasonic transducer;

FIG. 25 is a side cross-sectional view of a closed-configuration ultrasound-assisted debubbling apparatus in accordance with the third non-limiting example embodiment;

FIGS. 26(a) and 26(b) are graphs illustrating the performance of the closed-configuration ultrasound-assisted debubbling apparatus shown in FIG. 25 compared with a standard arterial filter;

FIG. 27 is a bar graph illustrating the performance of the open-configuration ultrasound-assisted debubbling apparatus shown in FIG. 24 compared with a standard arterial filter;

FIG. 28(a) is non-limiting schematic diagram of an example oscillator/amplifier for driving a large-area ultrasonic transducer; and

FIG. 28(b) is a schematic diagram of another example oscillator/amplifier for driving a large-area ultrasonic transducer.

### DETAILED DESCRIPTION

The following description sets forth specific details, such as particular embodiments, procedures, techniques, etc. for purposes of explanation and not limitation. But it will be appreciated by one skilled in the art that other embodiments may be employed apart from these specific details. In some instances, detailed descriptions of well known methods, circuits, and devices are omitted so as not obscure the description with unnecessary detail. Moreover, individual blocks are shown in some of the figures. Those skilled in the art will appreciate that the function of the controller block may be implemented using individual hardware circuits, using software programs and data, in conjunction with a suitably programmed digital microprocessor or general purpose computer, using application specific integrated circuitry (ASIC), and/or using one or more digital signal processors (DSPs).

As explained in the background, one particularly advantageous application for the technology described in this application is in the context of extra corporeal blood (CPB) circuits. However, those skilled in the art will appreciate that this is a non-limiting example application and that the technology in this case may be applied to any fluid from which any type of bubble is to be removed from the liquid. Other example applications include removing bubbles from a photographic emulsion or from certain industrial components whose performance requires a substantially bubble-free fluid. Although air bubbles are a common example, the term “bubble” includes any type of gas dissolved in or otherwise embedded in a liquid.

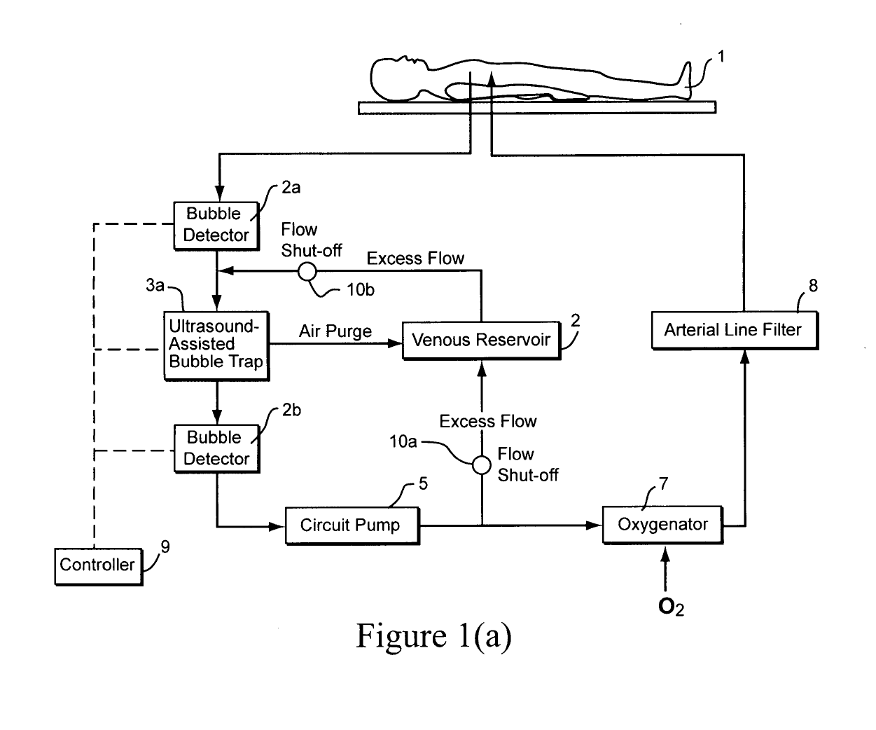

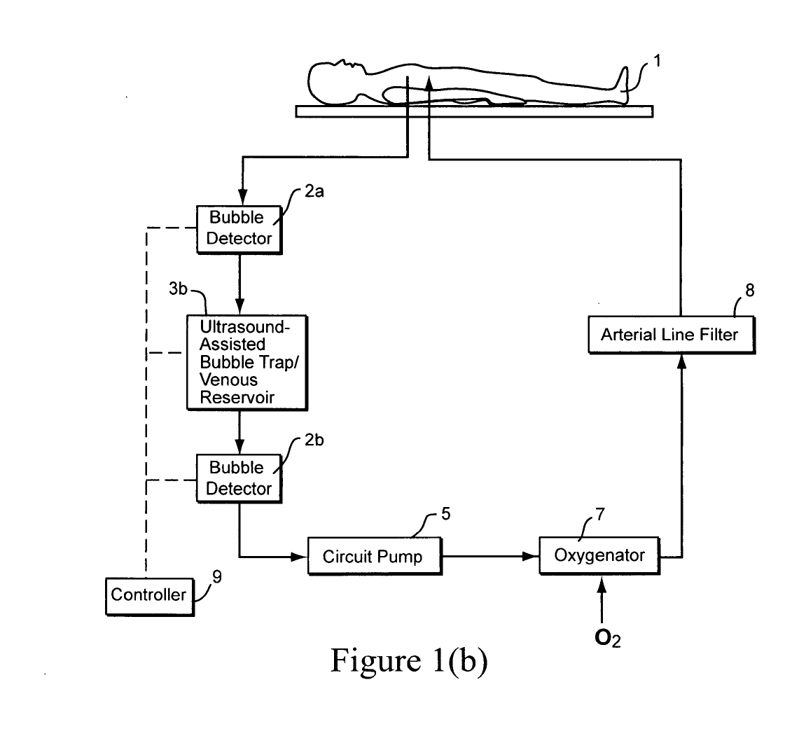

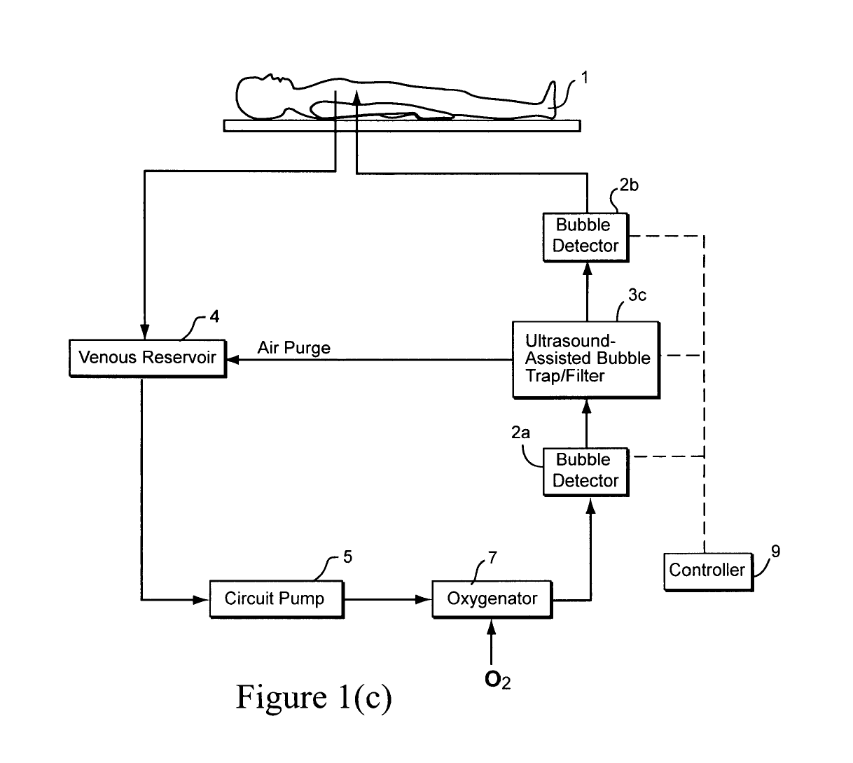

FIG. 1(a) is a non-limiting example of an extracorporeal blood (CPB) circuit in which gaseous emboli are removed. A patient 1 is shown coupled to the CPB circuit. Blood from the patient 1 is provided to a bubble detector 2a which detects the presence of bubbles in the blood and provides a signal to a controller 9. A suitable example bubble detector is an ultrasonic microemboli detector such as the EDAC® Quantifier from Luna Innovations Inc. The blood continues to an ultrasound-assisted bubble removing vessel or “trap” corresponding to a debubbling apparatus 3a. The debubbling apparatus 3a removes air bubbles and other gaseous emboli from the blood and vents them via an air purge line to a venous reservoir 2.

Blood from the debubbling apparatus 3a may be monitored by a second bubble detector 2b to determine if any bubbles remain in the blood. If bubbles are detected, the second bubble detector 2b notifies the controller 9 that bubbles remain in the blood and corrective action is taken to prevent additional bubbles from exiting the debubbling apparatus. The blood is provided through a circuit pump 5 which keeps the blood moving throughout the CPB circuit. The output fluid from the circuit pump 5 may be provided back to the venous reservoir 2 via a flow shut-off valve 10a. This shut-off valve is useful to maintain the correct volume of blood flow in the CPB circuit by removing excess blood. Blood may also be returned to the CPB circuit and to debubbling apparatus 3a from the venous reservoir via a second flow shut-off valve 10b. The flow shut-off valves may be controlled by the controller 9 or may be manually controlled.

When the flow shut-off valve 10a is closed, the blood in the circuit flows to an oxygenator 7 in which oxygen is infused into the blood. The oxygenated blood is then provided to an optional arterial line filter 8 which provides additional protection for the patient 1 from filters out gaseous and solid emboli. Details of non-limiting examples of the debubbling apparatus 3 will be described below in conjunction with subsequent figures.

The controller 9 receives information from the optional bubble detectors 2a and 2b, controls the ultrasound assisted bubble trap 3a, and may also control the shut-off valves. The controller 9 operates the ultrasound transducer in the debubbling apparatus 3 at an appropriate power level and frequency. When no bubbles are detected by bubble detector 2a or 2b, the controller 9 may optionally deactivate the debubbling apparatus 3. Alternatively, it may be desirable to operate the debubbling apparatus 3 as long as blood is flowing through the CPB circuit.

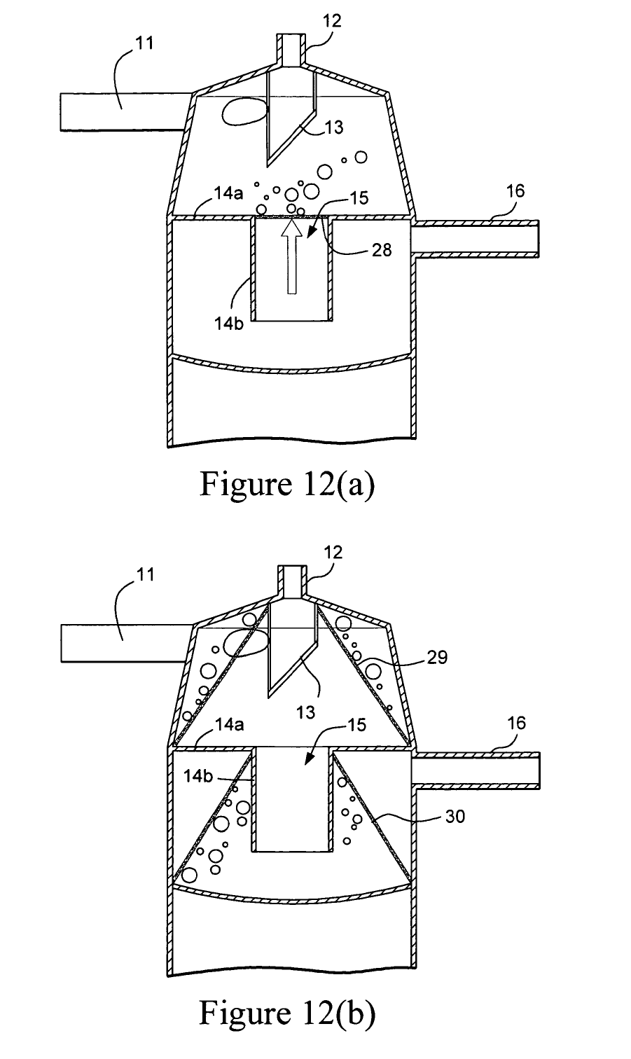

FIG. 1(b) is another non-limiting example CPB circuit in which gaseous emboli are removed. FIG. 1(b) is similar to FIG. 1(a) except that the ultrasound-assisted bubble trap is combined with the venous reservoir into one component 3b, while the configuration in FIG. 1(a) eliminates the venous reservoir from the main circuit loop. Both configurations are desirable in that they eliminate bubbles closer to their source which may have some clinical benefit in reduced inflammation due to platelet activation in the blood. The configuration in FIG. 1(b) may be more consistent with current practice in a CPB circuit than the configuration shown in FIG. 1(a) and therefore may be preferred. A more detailed non-limiting example of such a component 3b is shown in FIG. 11.

FIG. 1(c) is another non-limiting example CPB circuit in which gaseous emboli are removed. Here, the debubbling apparatus 3 is positioned on the arterial portion of the CPB circuit rather than on the venous side. Blood from the patient 1 is received at the venous reservoir 4 from which it is pumped by the circuit pump 5 to the oxygenator 7. The oxygenated blood from oxygenator 7 is then provided to bubble detector 2a, then to a debubbling apparatus implemented as a combined ultrasound-assisted bubble trap/arterial line filter 3c and bubble detector 2b before the blood is returned to the patient 1. This may be a desirable configuration because it removes gaseous from blood immediately prior to returning it the patient, and therefore, protects against small undetected leaks or other accidents that may occur downstream of the pump.

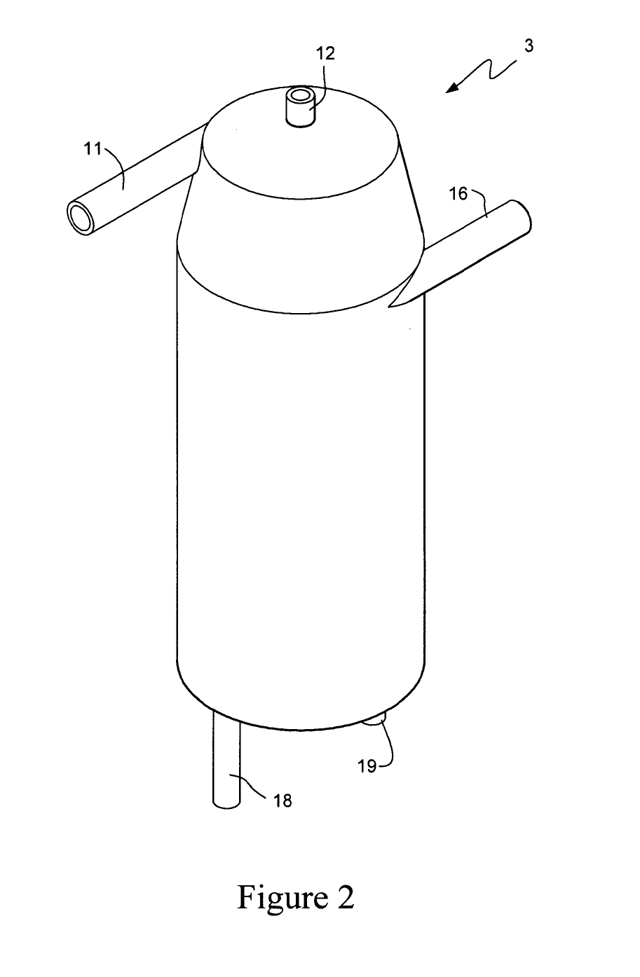

FIG. 2 is a front, perspective view of an ultrasound-assisted debubbling apparatus in accordance with a first non-limiting example embodiment. The debubbling apparatus 3 is a generally cylindrically-shaped vessel that includes a fluid inlet port 11 for receiving a fluid to be debubbled, such as blood, a fluid outlet port 16 for outputting the debubbled fluid, and a bubble outlet port 12 for exhausting bubbles from the vessel that have been removed from the fluid. An ultrasonic transducer (not shown here), which transmits an ultrasonic beam through the received fluid in the vessel to move those bubbles toward the bubble outlet port 12 as will be illustrated and described further below, may generate heat that can be damaging the fluid and/or to the transducer itself. Accordingly, the other end of the vessel may include a cooling fluid inlet 18 and a cooling fluid outlet 19 for circulating cooling fluid in a portion of the vessel to cool the vessel and an ultrasonic transducer mounted in (or near) the vessel.

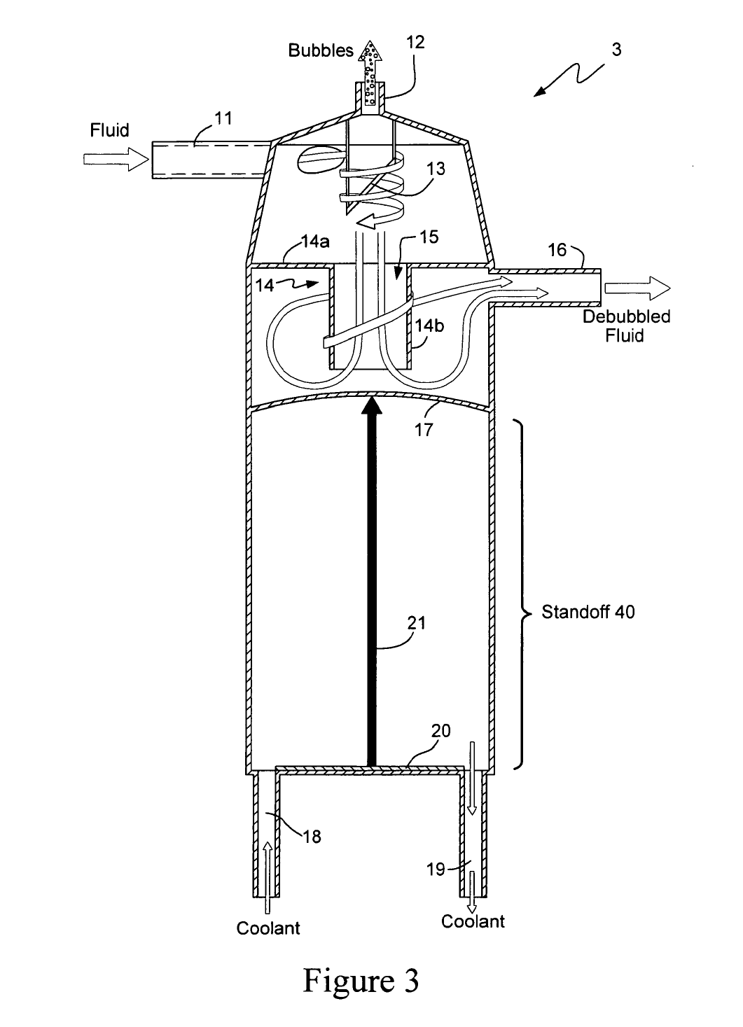

FIG. 3 is a cross-sectional view of the ultrasound-assisted debubbling apparatus in FIG. 2. The debubbling apparatus vessel is divided into three regions for ease of description: a fluid inlet region, a fluid outlet region, and an ultrasonic standoff region. Fluid enters the fluid inlet port 11 which preferably has a tangential orientation to the generally cylindrical shape of the vessel to encourage swirling flow of the blood inside the vessel as conceptually illustrated. Although the vessel is shown as being generally cylindrical, the vessel may be structured in other types of shapes. However, a cylindrically-shaped vessel is preferred because it also encourages swirling flow of the fluid which forces less dense particles such as air bubbles to the center of the vessel and coalesces them into larger bubbles that have sufficient buoyant force to rise to the top of the fluid inlet chamber where they may be removed via the bubble outlet port 12 as indicated. The fluid inlet chamber may be tapered as shown in FIG. 3 to direct the bubbles towards the bubble outlet port 12.

An ultrasonic transducer 20 is mounted at the other end of the vessel and generates an ultrasonic beam 21 that travels in a direction along a longitudinal axis of the vessel towards the fluid inlet region. The ultrasonic transducer 20 is preferably mounted within the vessel, but it may be mounted outside the vessel if desired. A non-limiting example of a suitable ultrasonic transducer is a lead zirconate titanate (PZT) crystal or another piezoelectric material that vibrates in response to an applied voltage. The transducer 20 is operated at a suitable power and frequency, e.g., by controller 9. A non-limiting example of power levels and frequency ranges for removing air bubbles from blood includes powers ranging from 1-190 W/cm² and frequencies ranging from 100 kHz to 10 MHz. Of course, these ranges are only examples and other frequencies and powers may be used. The ranges also depend on the application, the flow rate of the fluid, the viscosity of the fluid, and the size of the bubbles to be removed. The ultrasonic beam 21 carries momentum that is transferred to the air bubbles upon reflection or absorption of the ultrasonic beam which moves the bubbles towards the top of the fluid inlet region where they are withdrawn from the bubble outlet port 12.

The fluid inlet region is separated from a fluid outlet region by a barrier structure indicated generally at 14 that includes a first barrier portion 14a and a second barrier portion 14b. The barrier structure 14 may be made of biocompatible plastic such as polycarbonate but other materials may be used. The purpose of the barrier 14 is to block the bubbles from moving along with the fluid to the fluid outlet port 16 while at the same time still providing a path for the received fluid to reach the fluid outlet port 16. The first portion of the barrier 14a is a substantially horizontal surface with an opening 15 in the surface sufficiently aligned with the ultrasonic beam 21 so that at least a substantial portion of the ultrasonic beam energy reaches the fluid inlet region. The opening 15 in a preferred non-limiting example embodiment is circular so that the second barrier portion 14b is a cylinder substantially at a right angle to the first barrier portion 14a. The second barrier portion 4b confines the bubbles so that they are within the ultrasonic beam which maximizes the amount of power available to push the bubbles upward toward the bubble outlet port 12. Preferably, the dimensions of the opening 15 substantially match the cross-section of the ultrasonic beam 21 so that there is substantially uniform acoustic pressure across the opening 15 where the fluid passes from the fluid inlet region to the fluid outlet region. If the acoustic pressure is not uniform across the opening, bubbles may be able to pass through regions of the opening where the acoustic pressure is at a minimum.

Although the first and second barrier portions 14a and 14b are shown as perpendicular, they need not be and may be oriented in any position that transfers a substantial amount of the ultrasonic beam energy into the fluid inlet region while at the same time making it difficult for air bubbles to pass through the barrier opening into the fluid outlet region. Similarly, the shape of the barrier(s) and the opening need not be as shown, but instead can be any suitable shape that transfers a substantial amount of the ultrasonic beam energy into the fluid inlet region while at the same time making it difficult for air bubbles to pass through the barrier opening into the fluid outlet region.

The fluid inlet region also includes an acoustic reflection element 13 that redirects the ultrasonic beam 21 away from the fluid outlet region as will be described in further detail below. Preferably, the reflection element 13 directs the ultrasonic beam 21 in such a way so as to maximize the amount of acoustic radiation force that is directed up toward the bubble removal port 12 in order to move bubbles in the blood in that direction.

An acoustic window 17, essentially a fluid barrier, is made of acoustically transparent material separates the ultrasonic transducer 20 from the fluid. Non-limiting examples of acoustically transparent materials include polystyrene or mylar. As illustrated in FIG. 3, the region of the vessel between the ultrasonic transducer 20 and the acoustic window 17 defines an ultrasonic stand-off region. Although not necessary, the acoustic window 17 may be shaped to either focus or defocus the ultrasound beam 21 so that the beam profile substantially matches the dimensions of the opening 15 in the barrier 14. Example focusing properties of the acoustic window 17 are described in further detail below.

One or more dimensions of the ultrasonic standoff region may be sized/shaped in order to increase or maximize the amount of acoustic energy transmitted into the fluid inlet region. One non-limiting example is to angle the sidewalls to serve as an acoustic collimator or by adjusting the distance between the ultrasonic transducer 20 and the acoustically transparent medium 17 so that the position of the acoustic window 17 matches the near-field/far-field transition of the ultrasonic beam 21 where the sound wave is at a maximum. In high-frequency ultrasonic transducers, e.g., 1 MHz and up, this distance may be fairly large, e.g., on the order of 1 meter for a 1 cm beam width, which may have an attenuating effect on the ultrasonic beam 21. In this case, shortening the distance between the transducer and the acoustically transparent medium so that the fluid barrier is entirely within the near field may result in better performance.

To produce sufficient radiation force to drive microbubbles upward against the fluid flow, it may be desirable to drive the ultrasonic transducer at high powers that generate significant heat. In such a case, it would be desirable to cool the ultrasonic transducer 20. The ultrasonic standoff region receives cooling fluid through a coolant inlet 18 which is circulated in the ultrasonic standoff region and removed via a coolant outlet 19. Water is a non-limiting example coolant. Alternatively, externally applied coolant may be used to cool the walls of the standoff region. The ultrasonic standoff cooling fluid prevents the ultrasonic transducer 20 from overheating when operating at high powers required to force bubbles upwards at high flow rates. In CPB circuits, for example, the cooling water also prevents damage to blood from the heat generated by the ultrasonic transducer 20.

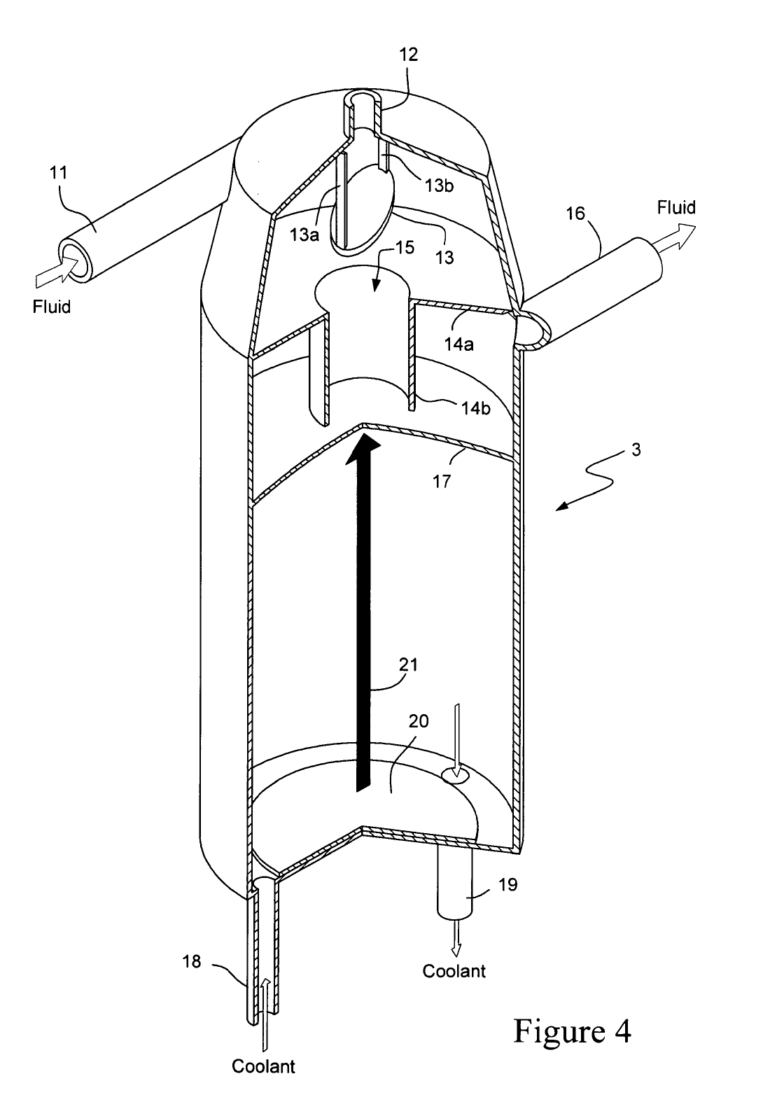

FIG. 4 is a three dimensional, perspective, cross-sectional view of the ultrasound-assisted debubbling apparatus of FIG. 2. This perspective cross-sectional view shows how the acoustic reflector 13 may be mounted inside the vessel using mounting members 13a and 13b. The perspective view also shows the barrier 14 with its first and second portions 14a and 14b which together form a cylindrical opening 15 that permits the fluid from the fluid inlet region to reach the fluid outlet region.

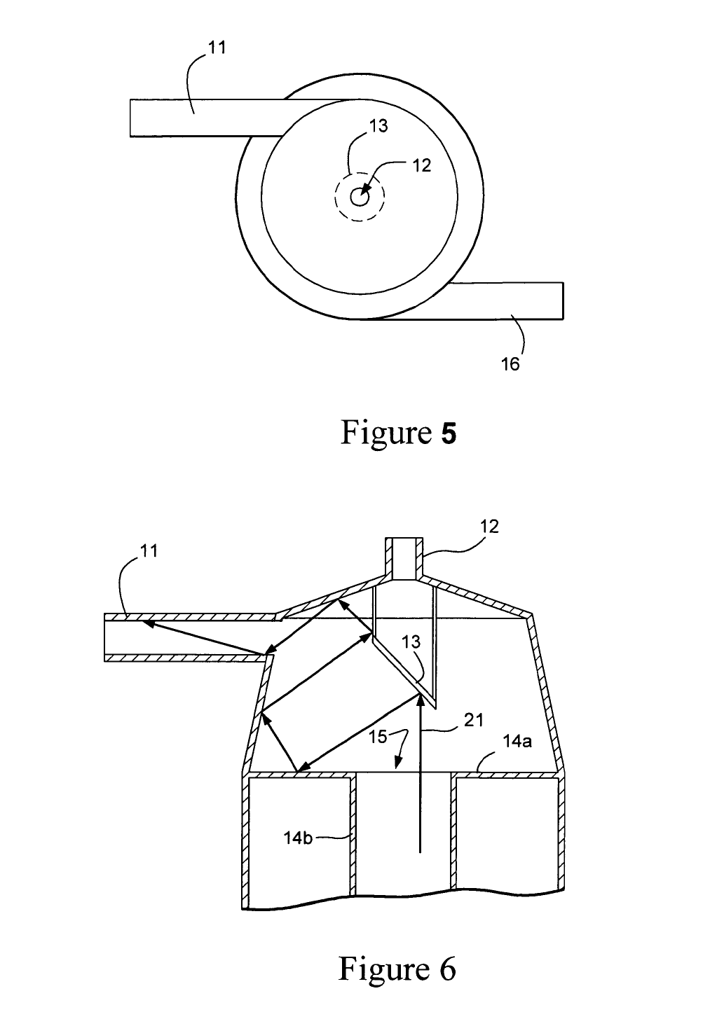

FIG. 5 is a top view of the debubbling apparatus in FIG. 2 and highlights the preferred, although not essential, tangential positioning of the fluid inlet port 11 and fluid outlet port 16 with respect to the vessel body to facilitate swirling flow of the fluid.

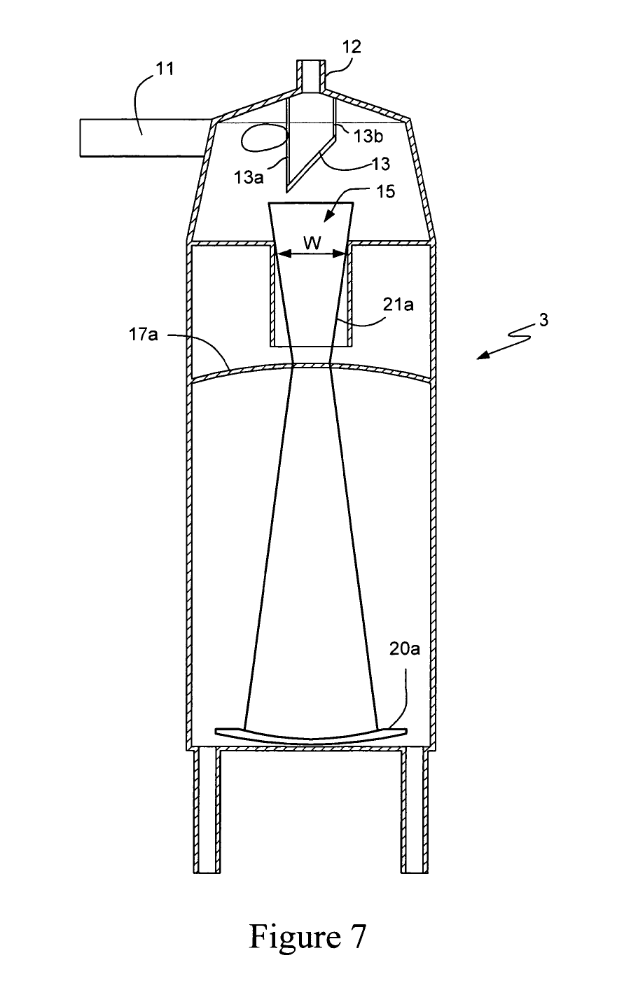

FIG. 6 is a partial cross-sectional drawing of the debubbling apparatus in FIG. 2 that illustrates how the acoustic reflector 13 redirects the ultrasonic beam 21 away from the opening 15 between the fluid inlet region and the fluid outlet region. The acoustic reflector 13 is angled in this non-limiting example so that the ultrasonic beam is reflected onto the first barrier portion 14a which reflects the beam toward the sidewalls of the vessel and then up to the top of the fluid inlet region, thereby pushing the air bubbles in the same upward direction toward the bubble outlet port 12.

After each reflection, some of the acoustic energy of the ultrasonic beam is transmitted into the reflecting material so that energy of the beam dissipates. Although after multiple reflections some acoustic energy may be directed downward through the opening 15, at that point, the energy of this multiply reflected beam will be substantially less than the energy of the incident ultrasonic beam coming up through the opening 15. In a preferred non-limiting implementation as shown, the reflector 13 is angled toward the fluid inlet port 11 so that the reflected acoustic energy hits bubbles immediately upon entering the apparatus so that the acoustic radiation force has more time to force bubbles upward toward the bubble outlet port.

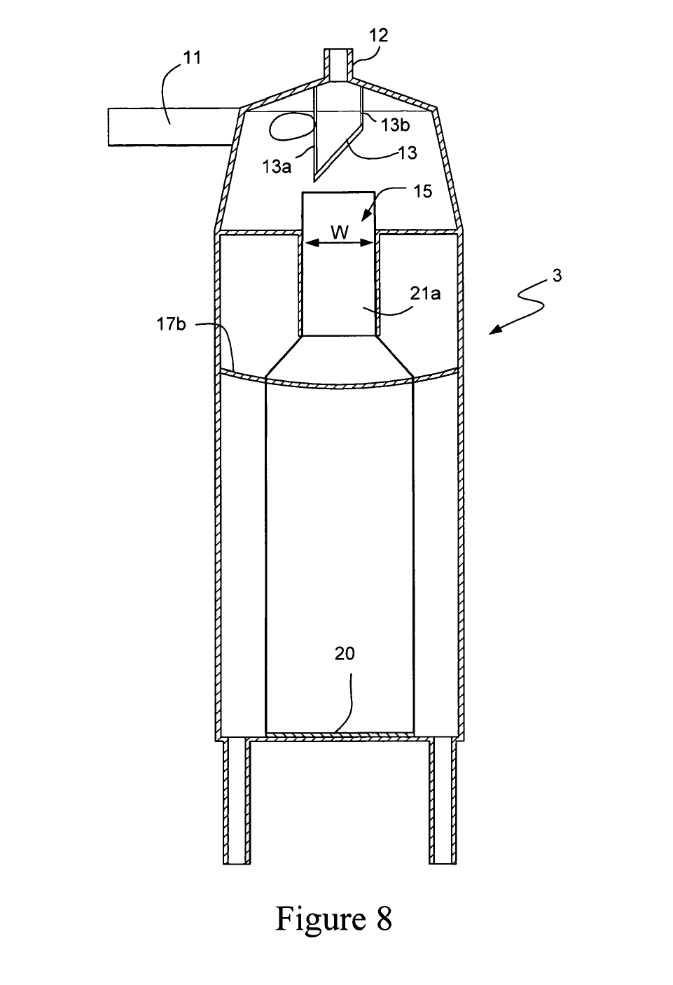

FIG. 7 is a cross-section of the ultrasound-assisted debubbling apparatus of FIG. 2 showing a non-limiting example embodiment with a curved ultrasonic transducer for shaping the ultrasonic beam. The ultrasonic transducer 20a is curved so as to focus the ultrasonic beam 21 towards the opening 15. As a result, the beam is more tightly collimated upon entering the fluid outlet region. In addition in this example embodiment, the acoustic window 17a is shaped so as to defocus the beam. The beam diameter of the defocused beam increases to match the width W of the opening 15 between the fluid inlet and fluid outlet regions.

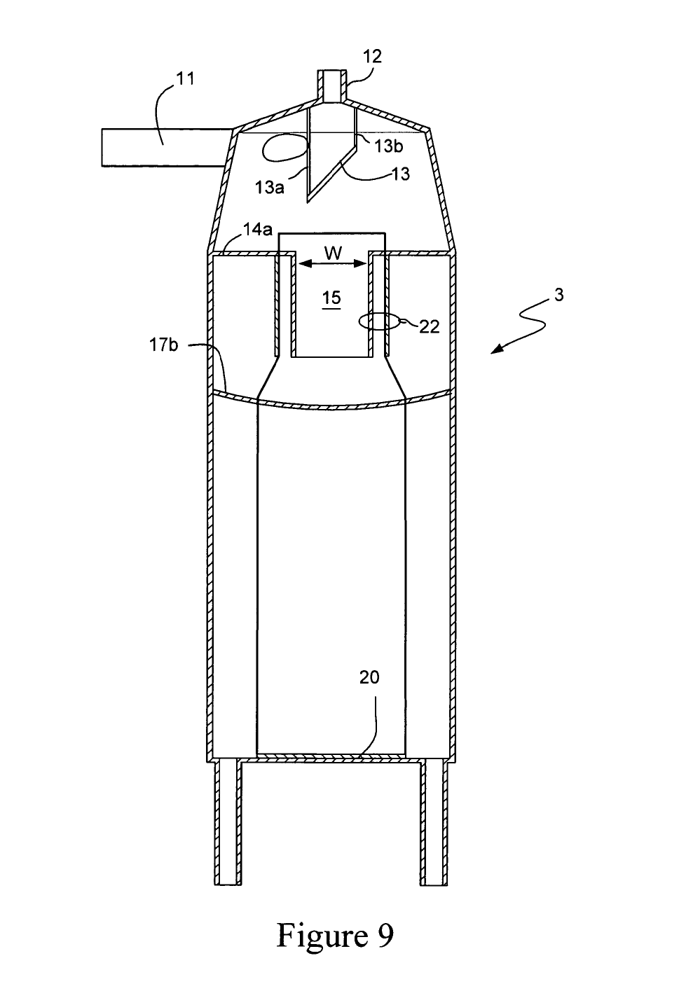

FIG. 8 is a cross-sectional view showing an alternative example embodiment of the debubbling apparatus in FIG. 2 showing a non-limiting example embodiment with a differently-shaped barrier separating an ultrasonic stand-off region from fluid regions in the vessel. The transducer 20 is not focused, and the acoustic window 17b is shaped to focus the ultrasonic beam 21. Note in this example, the beam 21 is wider than the opening 15. The shape of the acoustic window 17b is such that it focuses the acoustic beam 21 to substantially match the dimensions of the opening 15.

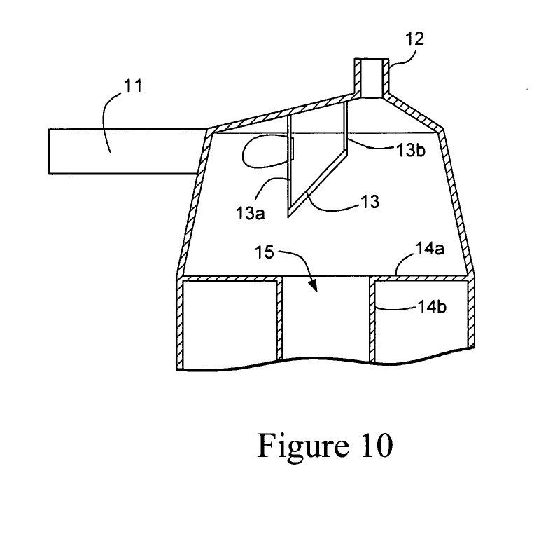

FIG. 9 is a cross-sectional view of the debubbling apparatus in FIG. 2 showing a non-limiting example embodiment with the barrier structure between the fluid inlet and fluid outlet ports having a concentrically-shaped portion. FIG. 9 is similar to FIG. 8 in that the ultrasound beam is wider than the opening 15. However, the second portion of the barrier 14, here shown at reference numeral 22, includes concentric cylinders. The acoustic window 17b is shaped to focus the beam so that it matches the width of the external concentric cylinder of the concentric barrier 22. In this way, bubbles that pass down through the opening 15 must travel perpendicularly to the ultrasonic traveling wave contained within the ultrasonic beam 21 in order to pass to the fluid outlet port 16. Consequently, less ultrasonic force is required to push the bubbles up, and any bubbles with sufficient energy to enter the fluid outlet region can be trapped between the two concentric cylinders of barrier 22.

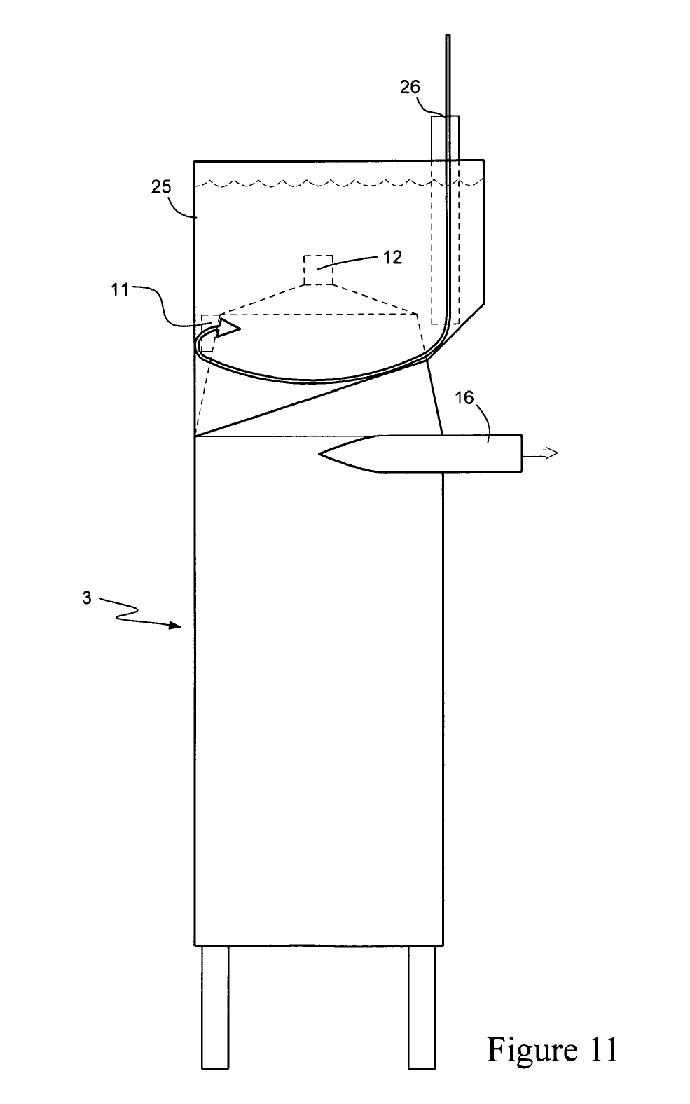

FIG. 10 is a partial cross-sectional view of the debubbling apparatus in FIG. 2 showing an alternative example embodiment where the bubble outlet port 12 is in a different location. Specifically, the bubble outlet port 12 is off-center from a central longitudinal axis of the debubbling vessel so that the outlet port is not directly above the acoustic reflector 13. As a result, the mounting members 13a and 13b will not block air from the reaching the bubble outlet port if they are constructed of a single cylindrical wall instead of two or more posts.

FIG. 11 is a side view of an alternative example embodiment for delivering fluid to the debubbling apparatus shown in FIG. 2. A container or bag is used as a reservoir 25 to receive and store fluid to be debubbled. The fluid to be debubbled is received from an inlet 26 and the pressure gradient within the CPB circuit pulls the fluid into the fluid inlet port 11. This configuration is an example for implementing the debubbling apparatus in the CPB circuit configuration shown in FIG. 1(b).

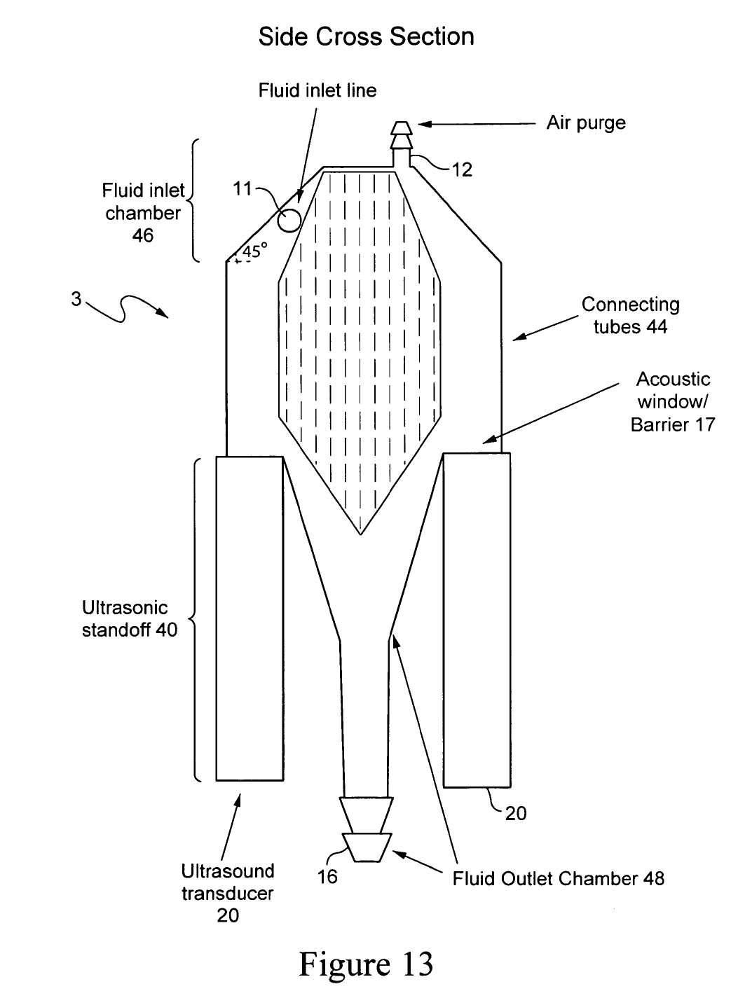

As is evident from the above description, the ultrasonic-assisted debubbling apparatus 3 includes multiple features that facilitate bubble removal from the fluid, which enhances the efficiency and reliability of the bubble removal process. Another bubble removal feature that may be used is one or more porous meshes to mechanically trap the bubbles or create barriers to the bubbles' movement within the vessel. FIG. 12a shows a non-limiting example embodiment where a porous mesh 28 is placed over the opening 15. The mesh 28 helps filter out bubbles from the fluid moving from the fluid inlet region to the fluid outlet region. In addition, the ultrasonic radiation force from the ultrasound beam can push the bubbles which are trapped in the porous mesh out of the mesh and back up toward the bubble outlet port 12. In other words, the ultrasound can “clear” trapped bubbles in the mesh.

Another alternative example mesh embodiment shown in FIG. 12b includes two conical mesh structures 29 and 30. The first conical mesh 29 structure is mounted in the fluid inlet region, and the second conical mesh structure 30 is mounted in the fluid outlet region. These meshes 29 and 30 are oriented at a substantial angle to the first barrier portion 14a. This substantial angle away from horizontal increases the surface area of the mesh, increasing the number of particles and bubbles that can be trapped without clogging the porous mesh and stopping flow.

Recall from the background that at higher flow rates in some applications, e.g., bubble removal from blood, the opening between the fluid inlet chamber and the fluid outlet chamber is preferably larger in order to slow the flow velocity down. But this larger opening requires an ultrasonic beam with a fairly large diameter and a high power density within the opening. Although a large-area transducer that can generate beams of this diameter and power is described in a second implementation of the second embodiment below, large-area transducers may be difficult to produce and are also prone to failure due to the multiple vibrational modes generated in over the larger surface area.

The first implementation of the second example embodiment includes an ultrasound-assisted debubbling apparatus that uses an array of smaller ultrasonic transducers, each ultrasonic transducer in the array propagating traveling ultrasound waves through one of an array of conduits (channels) that couple the fluid inlet chamber to the fluid outlet chamber of an ultrasound-assisted debubbling vessel. The channels may be implemented by producing an array of openings between the fluid inlet chamber and fluid outlet chamber. The fluid inlet chamber may be large enough to accommodate the transducer array. But in some applications, like blood filtering, this extra chamber size may not be desirable, as extra fluid volume results in greater hemodilution of blood and greater use of transfused blood during bypass surgery. For such applications, another example embodiment may be more suitable in which a small fluid inlet port is connected with a small fluid outlet port via an outer ring of ports in which a traveling ultrasonic wave pushes bubbles back toward the fluid inlet port.

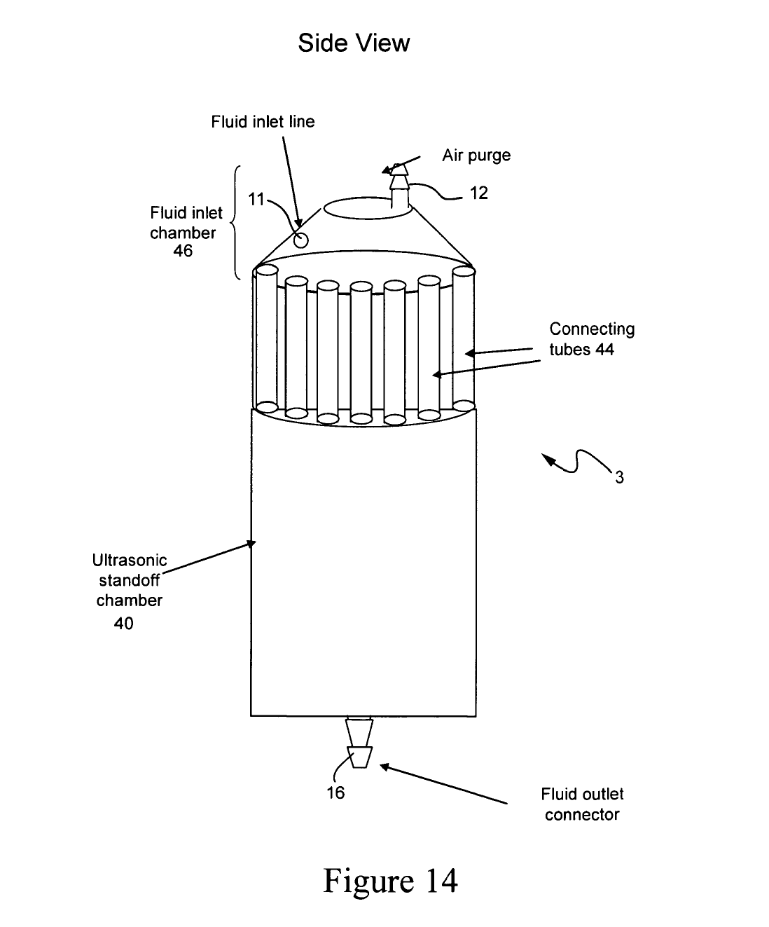

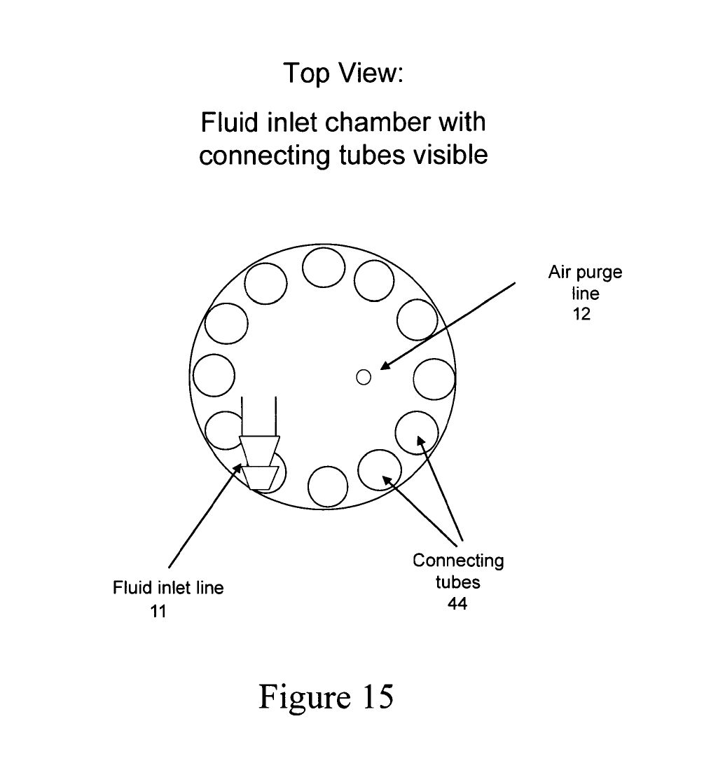

FIGS. 13 and 14 are side cross-sectional and side views respectively of an ultrasound-assisted debubbling apparatus in accordance with the first implementation of the second non-limiting example embodiment. A cylindrical fluid inlet port with an inlet 11 is positioned as shown so that fluid enters the top fluid inlet chamber 46 tangentially to initiate swirling fluid flow around the outside of the inlet chamber 46. During the swirling fluid flow, small bubbles coalesce into larger bubbles and bubbles with sufficient buoyant force rise to the top of the device and exit it through the air purge line 12. A gas-permeable membrane (not shown in FIG. 13) may be placed at this purge line to so that air can escape without removing fluid, or the purge line 12 may return the fluid/air mixture further upstream of the patient so that bubbles, while not completely removed from the bypass circuit, never reach the patient.

The outer boundary of the bottom of the fluid inlet chamber 46 interfaces with a circular array of conduits which in this example are connecting tubes 44 that transmit fluid from the inlet chamber 46 to a fluid outlet chamber 48. Smaller bubbles mixed in the blood leave the fluid inlet chamber 46 through connecting tubes 44 in a downward direction. Each tube 44 is also a conduit to direct an ultrasonic beam traveling in an upward direction toward the fluid inlet chamber 46. The top of the fluid inlet chamber 46 is preferably angled so that this ultrasonic beam is redirected toward the center of the chamber 46 instead of reflecting back down the connecting tube 44. By directing the ultrasonic beam toward the center of the chamber 46, acoustic reflections that could dissipate the strength of the upwardly directed ultrasonic beam are eliminated or at least substantially reduced. The angled design of the fluid inlet chamber 46 also produces a solid core in the center of the device which minimizes the fluid volume of the device. Doing so reduces the total volume of blood outside the patient during bypass surgery. In addition, the top of this channel may be angled to reflect ultrasound waves travelling up the connecting tube away from these tubes so that the reflections do not dissipate the radiation force used to debubble the fluid.

As one example only, the fluid inlet chamber 46, connecting tubes 44, and fluid outlet chamber 48 may be made of a biocompatible plastic such as polycarbonate or acrylic.

The tubes 44 that connect the fluid inlet chamber 46 to the fluid outlet chamber 48 each have a hole at the side the of the tube at or near the bottom of the tube that allows fluid to enter the fluid outlet chamber 48. The bottom wall of each tube is made of an acoustically transparent material that allows sound waves to enter the tube. Below each connecting tube 44, there is an ultrasonic standoff region 40 surrounding the fluid outlet chamber 48 so that the ultrasonic beam from an ultrasound transducer 20 matched to the tube 44 may be focused to at least substantially match the dimensions of the tube 44. In one example, the ultrasonic standoff region 40 is a cylinder with fluid-filled tubes inside the cylinder located underneath the connecting tubes 44. At the bottom of each standoff region tube is an ultrasound transducer 20 that converts an electrical signal into an ultrasound wave or beam. This wave or beam propagates up each tube within the standoff device 40 into and up through the connecting tubes 44. The ultrasonic beams/waves impart an upward radiation force upon the bubbles in the fluid which forces the bubbles back up to the fluid inlet chamber 46 and out to the air purge line 12. Debubbled fluid exits through an opening of each connecting tube 44 at the bottom of the tube, where the fluid from each of the tubes collects through the fluid outlet chamber 48 (in this example funnel-shaped) and exits the device ultimately at 16.

Each connecting tube 44 may be separated from the ultrasonic standoff region using an acoustically-transparent window/barrier 17 made of polystyrene, mylar, polyethylene or another suitable low acoustic-loss material. The fluid inlet chamber, connecting tubes, and fluid outlet chamber may be made of a biocompatible plastic such as polycarbonate or acrylic, for example.

These ultrasound standoff tubes are separated from the connecting tubes by an acoustic window/barrier 17 that separates the standoff fluid from the blood. The ultrasonic standoff 40 is preferably made of a heat conducting metal such as aluminum or copper. The ultrasonic standoff region 40 also preferably provides a heat sink for heat generated by the array of transducers 20 during the conversion of electrical energy to mechanical energy. The standoff tubes may be filled with a cooling fluid to prevent the fluid being debubbled (e.g., blood) from getting too hot as well as to cool the ultrasonic transducers to prevent overheating and failure. This heat can be dissipated from the standoff region to the surrounding air or actively-removed from the standoff region by circulating fluid through it. Such heat dissipation protects the blood from excess heat. For example, radiating fins may be built into the walls of the standoff chamber 40 to facilitate heat removal from the device or the ultrasonic standoff fluid may be circulated out of the chamber to a cooling reservoir. Other cooling techniques may be used.

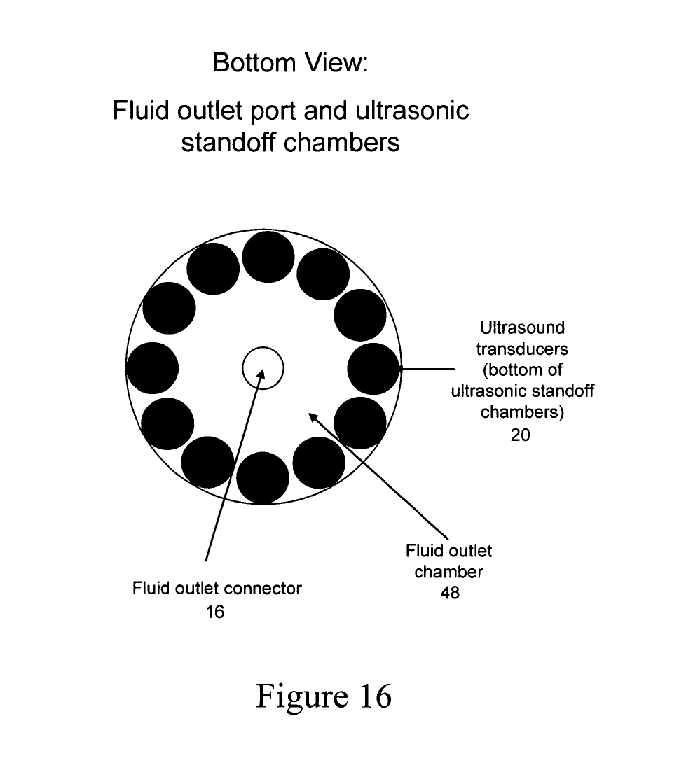

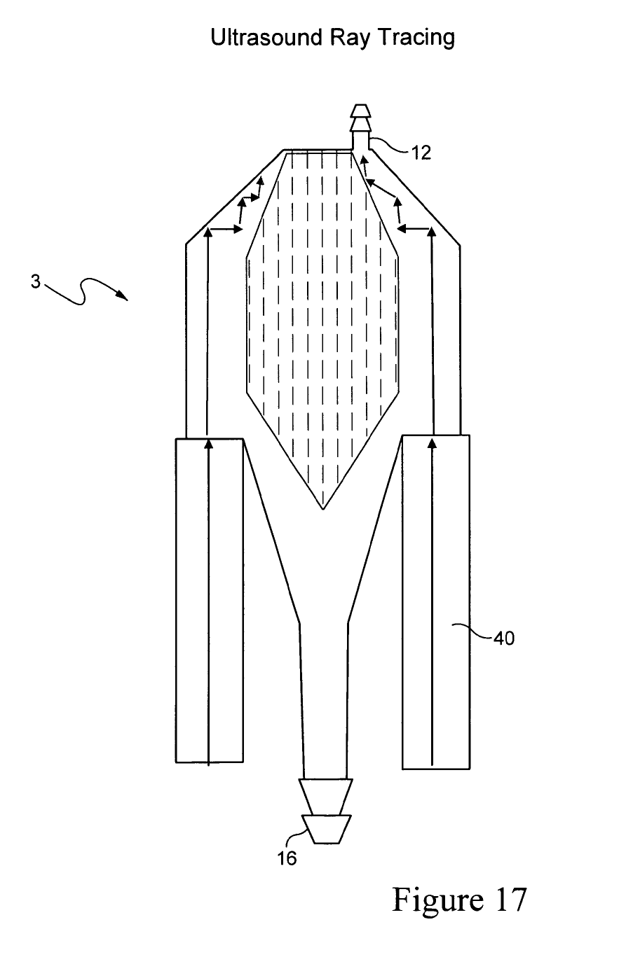

FIG. 15 is a top view and FIG. 16 is a bottom view of the ultrasound-assisted debubbling apparatus shown in FIG. 13. FIG. 15 shows the fluid inlet chamber 46 with the connecting tubes 44, fluid inlet line 11, and air purge line 12. FIG. 16 shows the fluid outlet port 48, ultrasonic standoff chambers 40, and ultrasonic transducers 20 at the bottom of the ultrasonic standoff chambers 40.

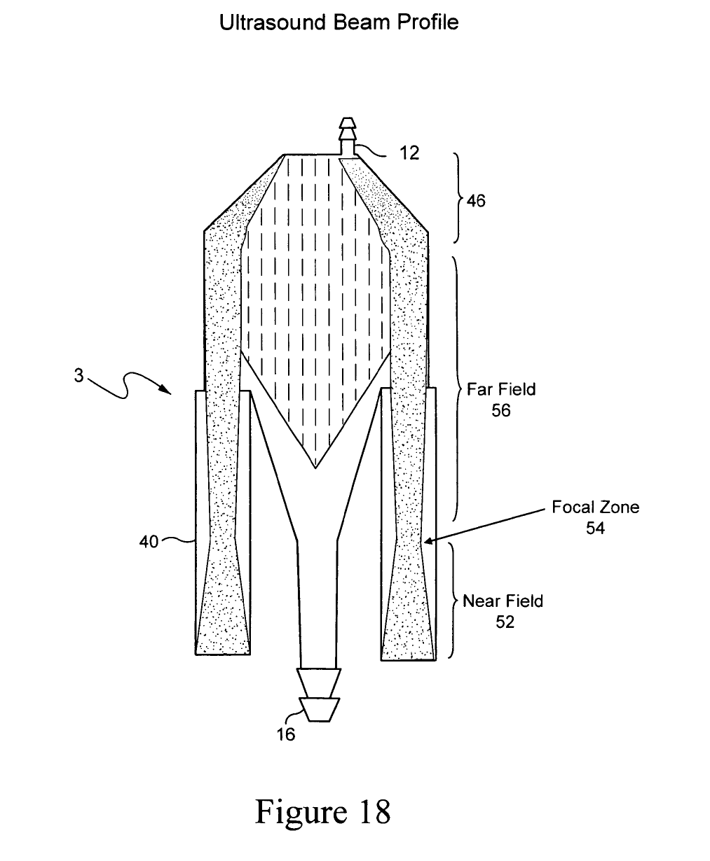

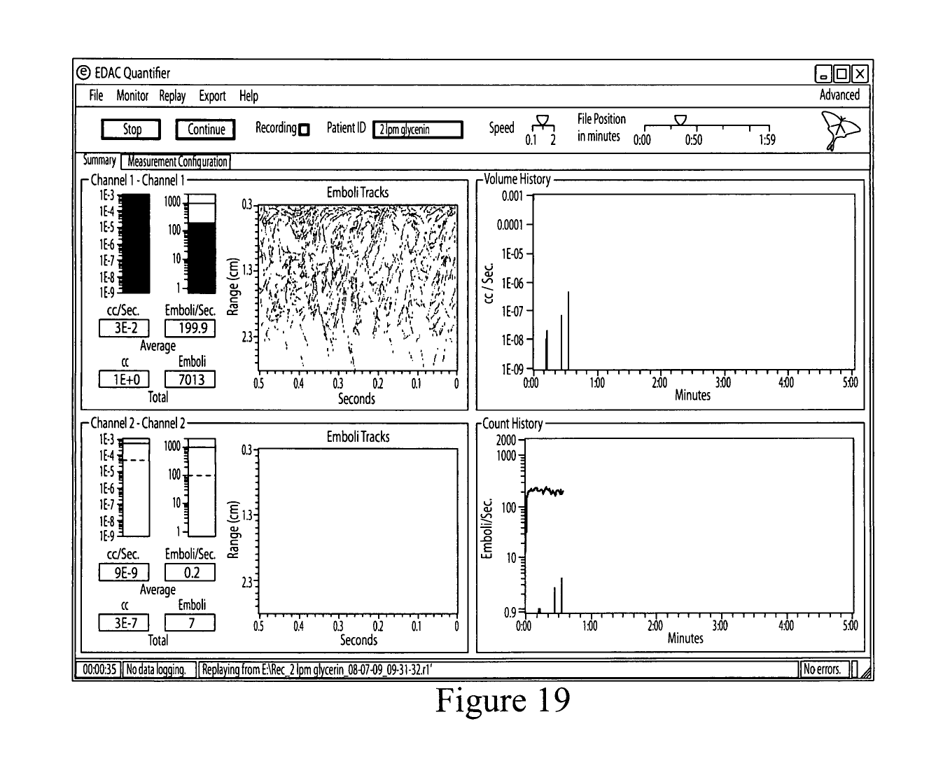

The connecting tubes 44 are insonified via traveling ultrasonic waves or beams. FIG. 17 shows a representative ultrasound beam tracing for a side cross-sectional view of the ultrasound-assisted debubbling apparatus shown in FIG. 13, and FIG. 18 shows an ultrasound beam profile. The ray tracing in FIG. 17 shows the direction of ultrasound beam propagation through the vessel 3, while the beam profile in FIG. 18 shows the dimensions of the ultrasound beam as it passes through the vessel 3. In the ray tracing, the ultrasound wave follows a straight-line path through an ultrasound standoff chamber 40, acoustic window/barrier 17, and connecting tube 44 until the wave is incident upon the angled walls of the fluid inlet chamber 46. The angle of these walls cause the ultrasound wave to reflect multiple times against the walls of the fluid inlet chamber. As one non-limiting example, the angle may be 45° or less with respect to the ultrasound wave. With each reflection, some of the ultrasonic energy is reflected and some is absorbed by the walls, so that the energy of the ultrasound wave is substantially reduced by the time it reaches the center of the fluid inlet chamber. Thus, little energy is reflected back in direction of fluid flow, maintaining a high-energy traveling wave through the connecting tube opposite the direction of fluid flow.

The beam profile in FIG. 18 shows that in the near field, i.e., the region of the ultrasound beam from the transducer face to the focal point, the ultrasound wave/beam largely matches the dimensions of the transducer 20 and only gradually narrows to a focal point N in the focal zone 52 according to the following equation:

$$N = \frac{{a}^{2}}{λ}$$

where N is the length of the near field from transducer to focal point, a is the radius of the transducer and λ is the wavelength of the ultrasound wave. At the focal point, the ultrasound wave enters the far field 56, i.e., the region of the ultrasound beam beyond the focal point where the beam begins to diverge. The angle of divergence is given by the following formula for a circular beam:

$$sin θ = \frac{1.22 λ}{d}$$

where θ is the angle of divergence, λ is the wavelength of the ultrasound wave and d is the diameter of the ultrasound transducer. Given N and θ from the above equations, the length L of the ultrasound standoff device 40 can be determined so that the width of the ultrasound beam as it enters its corresponding connecting tube 44 preferably substantially matches the width of the connecting tube 44 (labeled as w in the equation), as follows:

$$L = N + \frac{w - s}{2 tan θ}$$

where s is the beam width at the focal point N

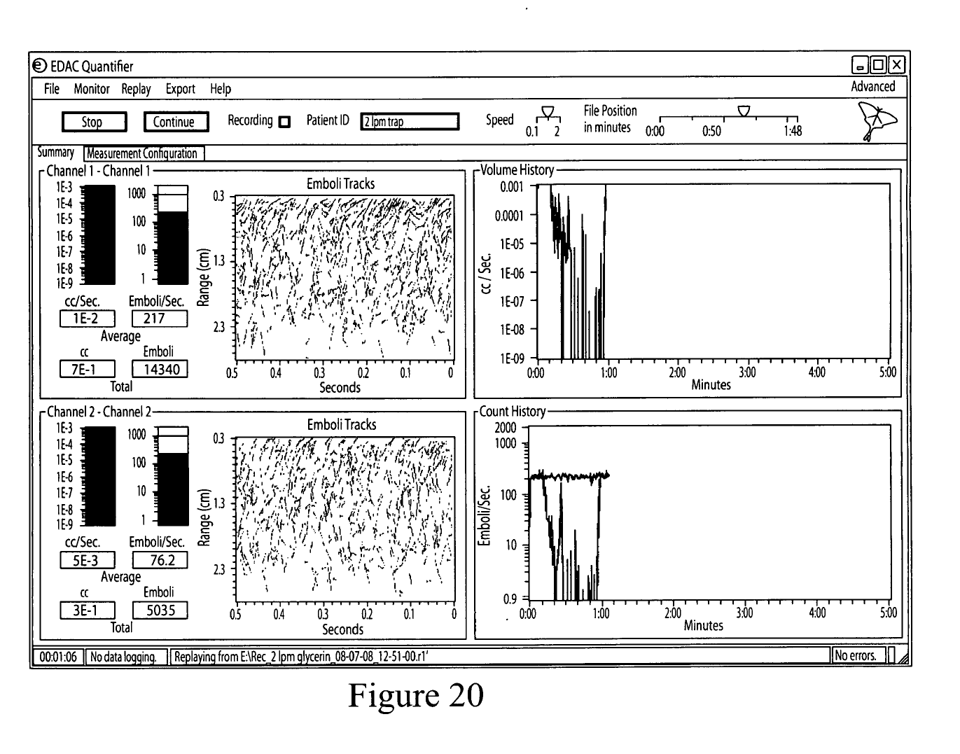

For testing purposes, a non-limiting single-channel debubbling apparatus was built and tested at flow rates up to 2 liters per minute. A 1.5″ diameter ultrasonic transducer was designed and used that produced a uniform ultrasonic beam through an opening 1″ in diameter between the fluid inlet chamber and the outlet chamber. During testing, an EDAC® Quantifier was used to determine if microbubbles were present prior to entering the test device and after exiting the test device. Results from one such test are shown in FIGS. 19 and 20.

In FIG. 19, bubble tracks are detected in blood before entering the microbubble filter (channel 1) but are eliminated from the bubble after the exiting the debubbling apparatus (channel 2). In FIG. 20, when the flow rate is increased from 2 to 4 liters per minute, the debubbling apparatus no longer removes bubbles from the blood (channel 2). In contrast to the single tube test device, a multi-tube debubbling device may be designed to allow operation at much higher flow rates without significantly increasing the volume of blood within the vessel 3. One non-limiting example of a higher flow rate is 7 liters per minute.

In contrast to the multi-transducer approach just-described, an alternative implementation for the second example embodiment that also achieves higher fluid flow rates through the debubbling apparatus is now described that employs a large area ultrasonic transducer. One example of an opening between the fluid inlet and outlet chambers so that the flow velocity is lower for a given higher volume flow rate, based on tests performed by the inventors, would be approximately three inches in diameter. An ultrasonic beam for this larger opening might need a power density within the opening on the order of 10 W/cm².

This estimate is based on experimental measurements and the following theoretical analysis of ultrasonic radiation force on an air bubble. The ultrasonic radiation force is produced by a difference in energy density on the incident side of the sound wave and the transmitted side, which is maximized for reflected sound waves. To a first order, the radiation force is given by the following equation for a spherical embolus:

$${F}_{US} = \frac{2 I π {r}^{2}}{c} \qquad ( 1 )$$

where I is the intensity of the ultrasound wave, r is the radius of the embolus, and c is the speed of sound of the transmission medium.

In a flowing viscous fluid, this force is balanced against viscous drag forces to produce the following equation of motion:

$$ρ V {x}^{″} = \frac{2 I π {r}^{2}}{c} - 6 π r μ ( {x}^{′} - {ν}_{f} ) \qquad ( 2 )$$

where ρ is the density of the embolus, V is its volume, μ is the viscosity of the fluid medium and ν_{f} is the velocity of the fluid medium. This equation (2) can be rearranged to form the following second order non-homogenous differential equation:

$${x}^{″} + \frac{9 μ}{2 ρ {r}^{2}} {x}^{′} = \frac{2 π {r}^{2}}{c} - 6 π r μ {ν}_{f} \qquad ( 3 )$$

This solution to this equation is:

$$x ( t ) = τ {ν}_{term} ( {e}^{\frac{- t}{τ}} - 1 ) + ( {ν}_{term} + {ν}_{f} ) t \qquad ( 4 )$$

where νₜₑᵣₘ is the terminal velocity of the embolus in a viscous fluid while subject to an ultrasonic radiation force, and τ is the time constant required for the embolus to reach its terminal velocity. The terminal velocity and time constant are given by the following formulae:

$$\begin{matrix}{ν}_{term} = \frac{I r}{μ c} & ( 5 ) \\ τ = \frac{2 ρ {r}^{2}}{9 μ} & ( 6 )\end{matrix}$$

For gaseous microemboli ranging in size from 5-500 microns, the time constant τ ranges from 2 nanoseconds to 20 microseconds. For lipid microemboli of the same size, the time constant ranges from 2 microseconds to 20 milliseconds. Given these small values, it can be assumed that the embolus reaches its terminal velocity instantaneously, in which case equation (4) reduces to:

x(t)=(μₜₑᵣₘ+ν_{f})t (7)

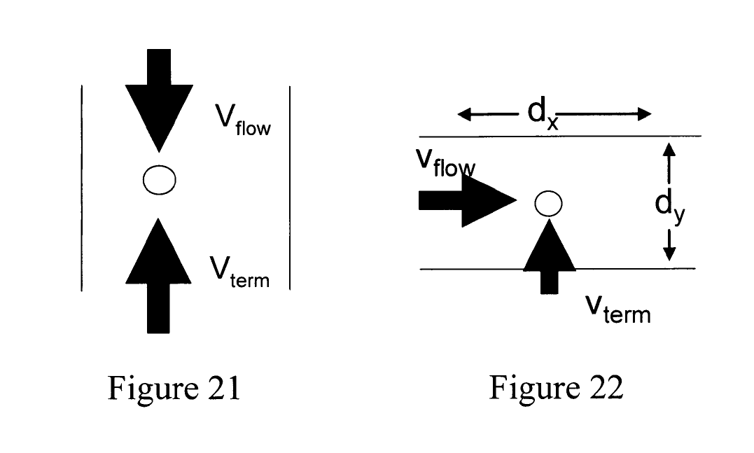

Equation (7) establishes that if the terminal velocity of the embolus subject to an ultrasound radiation force is greater than the velocity of the fluid flow, the embolus will be trapped in the ultrasound field. When the trap is arranged with the ultrasound field directed upward against the direction of fluid flow, as in FIG. 21, the terminal velocity of a 10 micron bubble in a 10 W/cm² acoustic field is 10 cm/s. At a maximum flow rate of 7 liters per minute, this would require a cross-sectional area of the de-bubbling apparatus vessel of 9 cm. At an example drive frequency of 2 MHz, the mechanical index of a 10 W/cm² ultrasound field is 0.25, almost 8 times below the FDA maximum of 1.9.

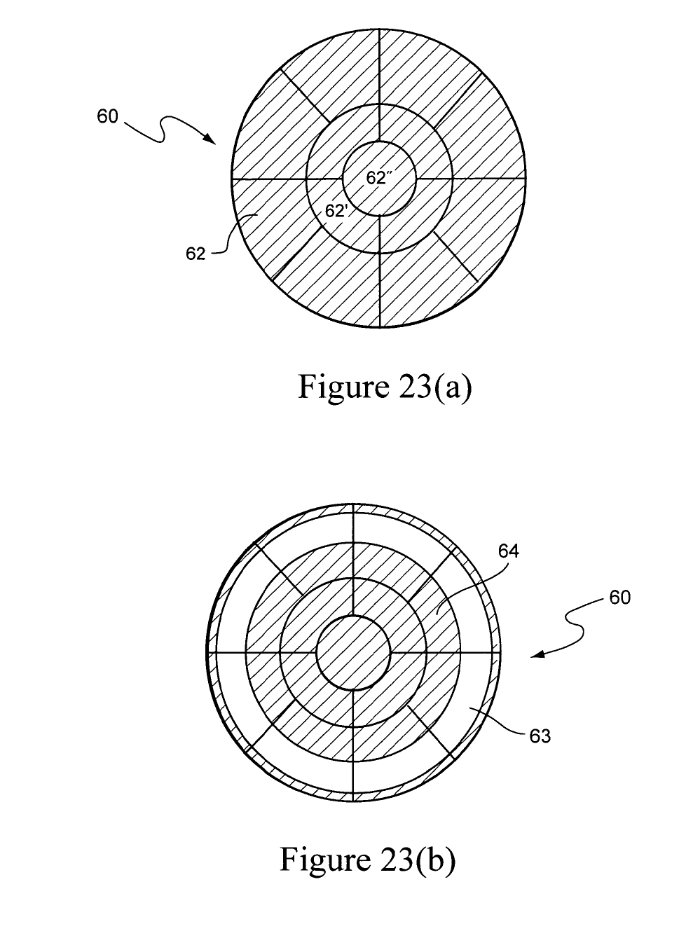

In an alternate configuration, the sound field may be directed perpendicular to the direction of fluid flow, as in FIG. 22. In this case, the embolus must be pushed outside the flowing fluid in the y-direction (d_{y}) before the embolus passes through the width of the ultrasound field (dₓ). At a maximum flow rate of 7 lpm in a standard a ⅜″ diameter tube, the diameter of the sound field would need to be 8 cm in order to push the bubble out of the flow, similar to the diameter of the sound field required for the upward directed field described above. This configuration has been disclosed separately in the works of Katz WO 2004/004571 A2 and Palanchon (“Acoustical bubble trapper applied to hemodialysis,” Ultrasound in Medicine and Biology 34:4 (April 2008), p. 681-684, and “Ultrasound based air bubble trapping system for hemodialysis,” Ultrasound in Medicine and Biology 32:5 (May 2006), p. 159). However, both groups have only tested their bubble traps at relatively low flow rates in the 100 mL/min range, where the bubble trap dimensions can be much smaller than the 8 cm tube required for operation at 7 lpm.

The upstream configuration of FIG. 21 over that of FIG. 22 is preferable because it is easier to scale this design to higher flow rates by making a wider ultrasound beam, and it is easier to integrate an air purge line into the debubbling apparatus. Without such an air purge line, bubbles can disrupt the ultrasound field and reduce the air removal efficiency.

The above analysis suggests that large-area ultrasonic transducers should be used to produce ultrasonic beams that match the dimensions of the debubbling apparatus. But ultrasonic transducers of this diameter and power are difficult to produce and are also prone to failure due to the multiple vibrational modes generated in over the larger surface. That is why the implementation described earlier using an array of smaller ultrasonic transducers may be attractive in some applications. On the other hand, the use of multiple ultrasonic transducers poses a number of practical problems. First, each ultrasonic transducer should produce approximately the same acoustic output power. If the power is too high in one ultrasonic transducer, the result will be damage to the blood, while if it is too low in one ultrasonic transducer, microbubbles will not be trapped. The output power of an ultrasonic transducer can be highly variable due to manufacturing variations in the piezoelectric crystal and mechanical differences in the way the transducer is mounted in the bubble trap. Second, small ultrasonic transducers have a far-field transition point that is much smaller than in larger ultrasonic transducers. At the far-field transition point, the ultrasound beam narrows to a small area and begins to diffract as if from a point source. As a result, the beam intensity is not uniform over a wide area, and complex beamforming is required to substantially match the beam intensity to the diameter of the conduits in the debubbling apparatus.

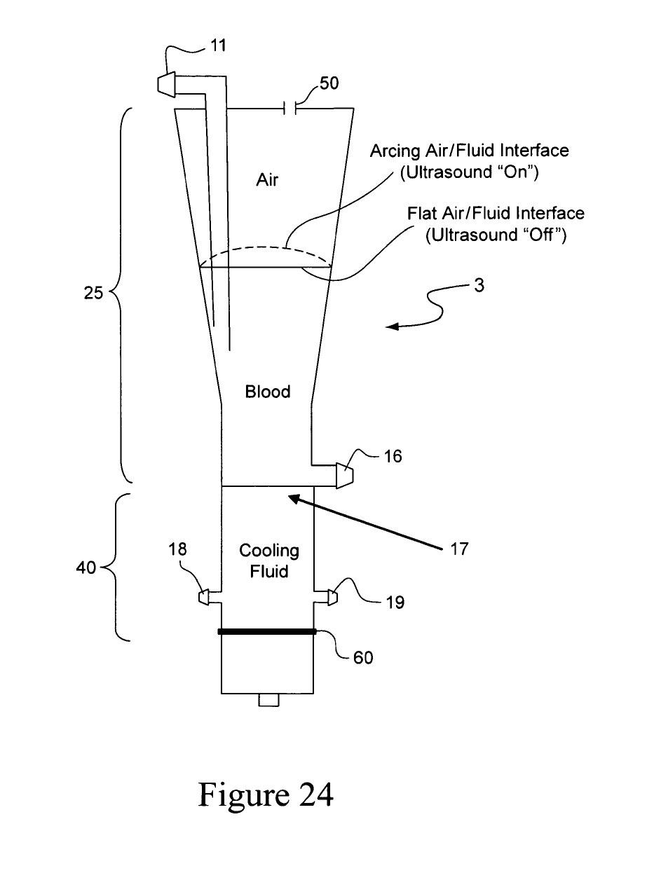

Given these difficulties, one or more large-area ultrasonic transducers may be more desirable in some applications. To prevent failure of a large-area ultrasonic transducer, it is preferably composed of multiple elements in tiled array. FIGS. 23(a) and 23(b) show a front face and back face of a non-limiting example segmented or tiled large-area transducer 60 for use in an ultrasound-assisted debubbling apparatus. The front face in FIG. 23(a) includes 13 transducer elements 62, of approximately the same area though at different sizes at each concentric ring 62, 62′, and 62′″. In this design, the entire front face may be metalized for connection to a positive electrode; this layer wraps around the edge of the transducer, so that positive electrode can be connected to the back side of the transducer. On the back side of the transducer shown in FIG. 23(b), the positive electrode is separated by an unmetallized concentric ring 63. The negative electrode 64 is deposited in the center of the back face. Each tile element is driven in phase by a single electrical signal.

With a large-area transducer, it is desirable to reduce the size of the debubbling apparatus in order to reduce the volume of fluid that the debubbling apparatus adds to the bypass circuit, so as to minimize the amount of blood outside the body of the patient. One example way to do this is to combine the fluid inlet chamber and the fluid outlet chamber into a single chamber with an inlet port at the top and an outlet port at the bottom as described in commonly-assigned application Ser. No. 12/129,985, entitled “Acoustically Enhanced Removal of Bubbles from a Fluid,” the contents of which are incorporated herein by reference.

From a theoretical standpoint, the height of the trap does not have a significant effect on bubble removal efficiency. Because the time constant z in equation (6) is on the order of microseconds, a bubble is trapped almost instantaneously, and a longer column should not improve the trapping efficiency. From a practical standpoint, however, the debubbling apparatus needs to be tall enough to provide a buffer volume for purging trapped bubbles from the chamber. If these bubbles are not quickly purged, they can disrupt the travelling sound wave and reduce the forward acoustic beam intensity. Therefore, there is a practical tradeoff between limiting the height of the trap to reduce prime volume and increasing the height to improve trapping efficiency. One example solution to this trade-off is to integrate the bubble trap into a venous reservoir, which does not add significant prime volume to the trap since the reservoir is already designed to store blood in the circuit.

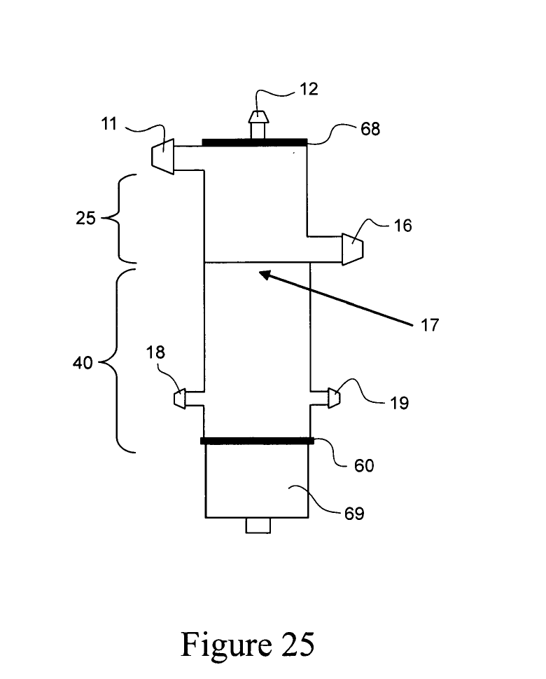

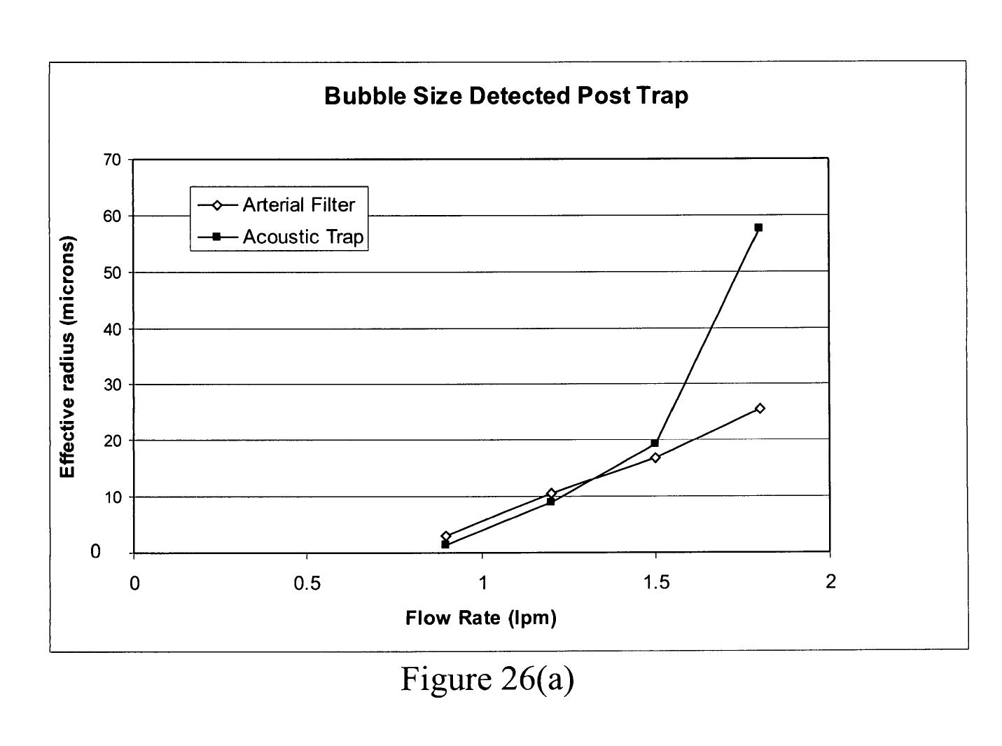

A non-limiting example of an integrated venous reservoir debubbling apparatus 3 is shown in FIG. 24. This implementation is an “open configuration,” in which the reservoir is open to air, producing a large fluid (e.g., blood)/air interface. In contrast, closed reservoirs employ a collapsible bag with no blood/air interface. A non-limiting example implementation of this closed configuration is described below in conjunction with FIG. 25. In FIG. 24, the fluid inlet port 11 enters an open shell reservoir 25 tangentially to and then extends vertically into the open shell reservoir which holds the fluid for debubbling. At the bottom of the reservoir 25, the dimensions of the reservoir 25 substantially match the diameter of the ultrasound beam from a large area transducer 60. Here, the fluid exits via the fluid outlet line 16. A pressure release valve 50 is positioned at the top of the reservoir to keep the reservoir at atmospheric pressure. A slight vacuum may be applied if desired to this valve to assist in bubble removal.

An acoustic window 17 is provided that may be made of polystyrene, polyethylene or another acoustically transparent material. In a standoff region 40, on the other side of the acoustic window 17, de-aired cooling fluid (e.g., water) circulates between the transducer 60 and the acoustic window 17 to at least reduce and preferably prevent heating of fluid (e.g., blood) in the reservoir 25. Cooling fluid (e.g., water) connection lines 18 and 19 are shown for circulating the cooling fluid.

The amount of acoustic energy reflected back toward the fluid outlet can be further minimized using the integrated design of FIG. 24 because the acoustic wave reflects off of an air/fluid interface. Assuming the fluid in this example is blood, FIG. 24 shows that the air/blood interface is relatively flat when the ultrasound transducer(s) is(are) off and relatively curved or arced when the ultrasound transducer(s) is(are) on. The force of the travelling acoustic wave produces an effect known as “acoustic streaming,” which produces a visible arc in the air/blood interface. Acoustic streaming dissipates the energy of the forward acoustic wave and minimizes reflected acoustic waves that reduce the radiation force on bubbles in the trap. This air/fluid interface may not be desirable in a bypass circuit, however, due to concerns relating to platelet activation and systemic inflammation.

Alternatively, it may be desirable to produce the debubbling apparatus 3 as a standalone unit without integrated venous reservoir as shown in FIG. 25. This closed configuration eliminates the large air interface within the open reservoir. Both of these reservoir designs can be placed in the bypass circuit in the position of the “Ultrasound-Assisted Bubble Trap/Venous Reservoir” shown in FIG. 1(b).

There is some concern that the air interface in an open shell reservoir configuration may contribute to platelet activation and systemic inflammation during bypass surgery, although open shell reservoirs are still in widespread use. The stand alone closed configuration in FIG. 25 eliminates that concern but still requires a purge line 12 at the top of the debubbling apparatus for removing purged air bubbles. This purge line may be routed back to the line running from the patient to the bubble detector 2a in FIG. 1(b), or to the venous reservoir 4 if the trap is placed on the arterial side of the bypass circuit, as shown in FIG. 1(c).

The purge line 12 may be integrated into the top of a reflecting element, at the top of the debubbling apparatus, similar to the non-limiting example shown in FIG. 3 (e.g., reflector 13) which is designed to minimize reflected acoustic waves by reducing the intensity of the forward travelling acoustic wave. A flat trap 68 shown in FIG. 25 also works as well so long as the debubbling apparatus is made of a plastic that matches well acoustically with the fluid so long as the plastic is acoustically attenuating or has an attenuating material such as tungsten powder added to it.

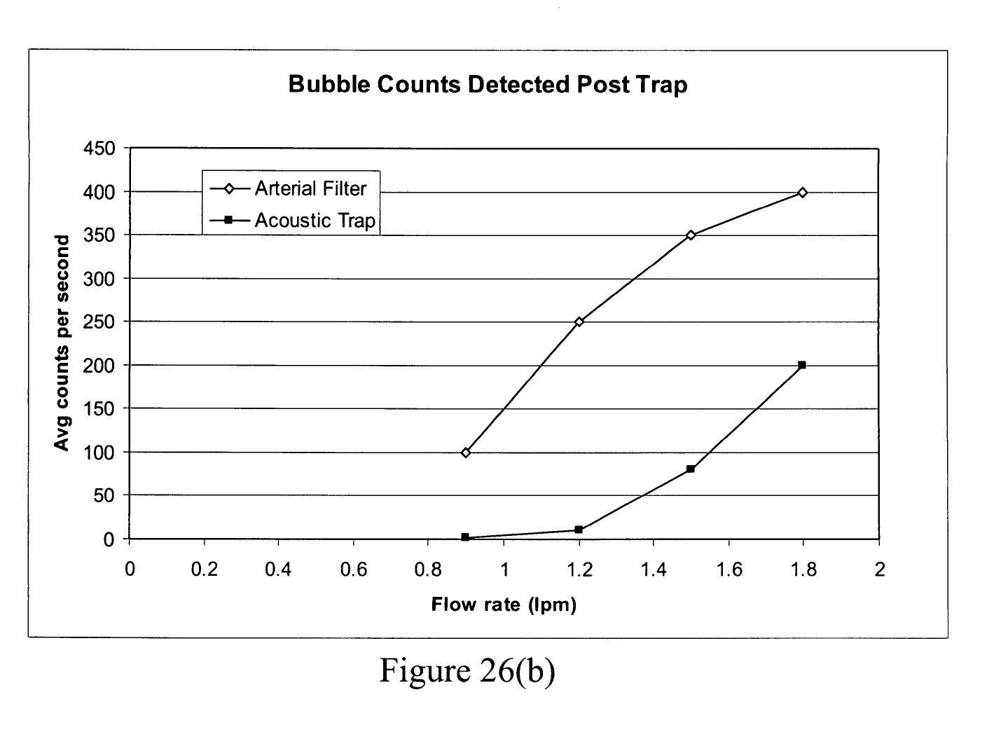

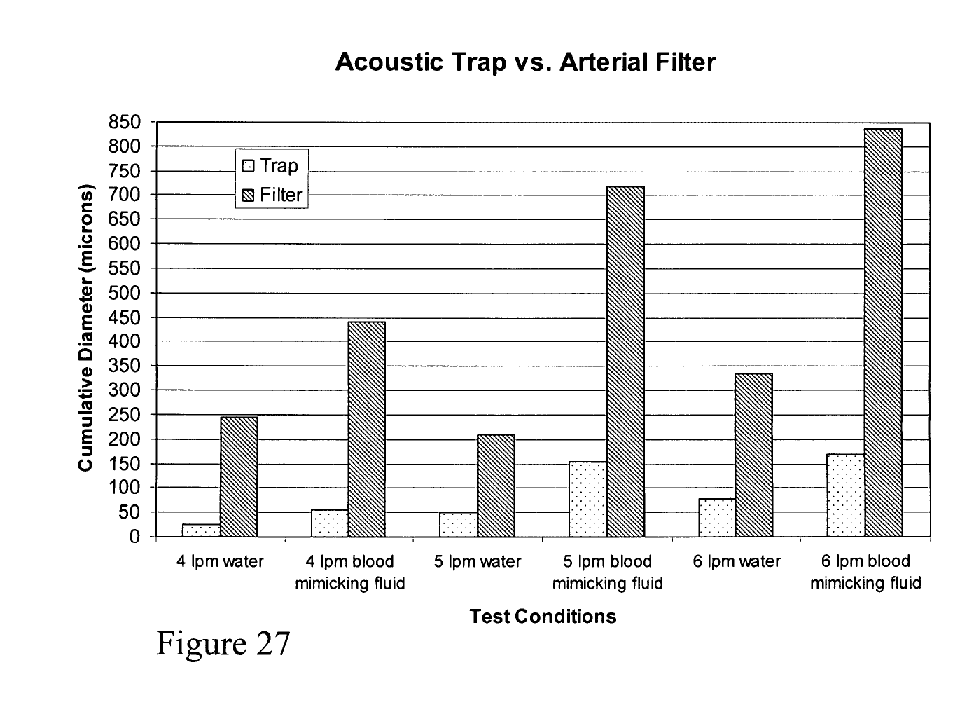

Test data comparing the air handling of example test versions of the open and closed configuration debubbling apparatus to arterial line filters are shown in FIGS. 26(a) and 26(b) and in FIG. 27. The graphs in FIGS. 26(a) and 26(b) pertain to the test closed-configuration debubbling apparatus (FIG. 24) which employs a single 1.5-inch transducer and is therefore only effective up to flow rates of 1.5-2 liters per minute. The bar graph in FIG. 27 pertains to the integrated open configuration test apparatus using a 3-inch diameter transducer that allows the trap to work better than an arterial filter at flow rates exceeding 6 liters per minute.

A concern with a debubbling apparatus is the potential of the ultrasound energy to damage blood due to the heat generated by the sound wave. While very little sound energy is directly absorbed by blood and converted to heat, a large amount of heat is generated at the transducer during the conversion of the electrical drive signal to a mechanical wave. Because this heat is concentrated within a small area close to the transducer face, that heat can raise the temperature around the crystal enough to cause hemolysis if this heat is not removed before reaching the blood. One solution is to employ a cooling fluid (e.g., water) standoff between the transducer face and the bubble trap as described above. If the standoff fluid circulates to a large water bath outside the trap, tests have shown that the circulating water never exceeds 30° C., even without cooling the circulating fluid.

With the standoff cooling fluid, care must be taken to prevent air bubbles from collecting on the acoustic window between the water standoff and the bubble trap, as these bubbles can block acoustic transmission into the debubbling apparatus. The amount of bubbles within the water standoff can be minimized by de-airing the standoff fluid prior to use in the debubbling apparatus and adding a surfactant that prevents bubbles from clinging to the acoustic window. Possible de-airing methods include the use of a Venturi pump to circuit the fluid in the standoff, and the addition of sodium sulfide to the standoff fluid. The Venturi pump provides a negative pressure the pulls air bubbles out of solution, while sodium sulfide binds strongly to oxygen, preventing air bubbles from combine out of solution.

The ultrasound transducers used in the debubbling apparatus shown in FIGS. 24 and 25 (as well as the apparatus shown in the other example embodiments) may be driven using a standard off-the-shelf RF amplifier that operates for example in the megahertz frequency range. However, standard RF amplifiers have an output impedance of 50 ohms which presents problem for large-area ultrasonic transducers which have lower input impedances. For example, the 1.5-inch diameter transducers used in the test structures noted above have an impedance of about 7 ohms. Larger-area transducers will have less impedance.

With an impedance mismatch this large, most of the input energy from the RF amplifier will be reflected back to the amplifier. To prevent such reflections, an impedance matching network or a transmission line network may be used.

An alternative is to design an RF amplifier whose impedance more closely matches that of the large-area transducer. One such approach has already been published in Lewis et al, “Development of a portable therapeutic and high intensity ultrasound system for military, medical and research use,” Rev Sci Instr. 79:114302 (2008). In this design, a TTL signal from an oscillator is used as an input to PIN drivers, which then drive an array of MOSFET amplifiers. The array of amplifiers is used to achieve the high currents needed to drive low impedance transducers.

An alternate approach described here combines an oscillator drive signal with an amplifier circuit. This oscillator/amplifier includes an adjustable continuous wave (CW) oscillator coupled to a push-pull power output stage. While this approach employs a similar oscillator and MOSFET array to the Lewis design, the device described in more detail below more closely resembles the output stages of high-power audio amplifiers.

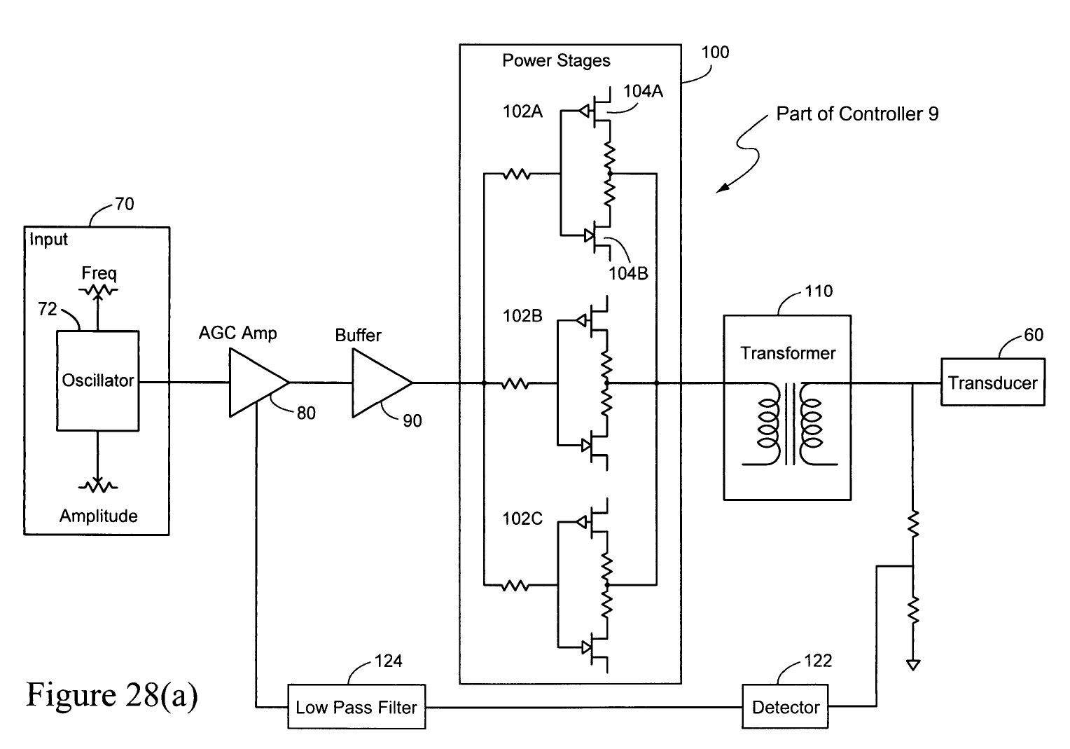

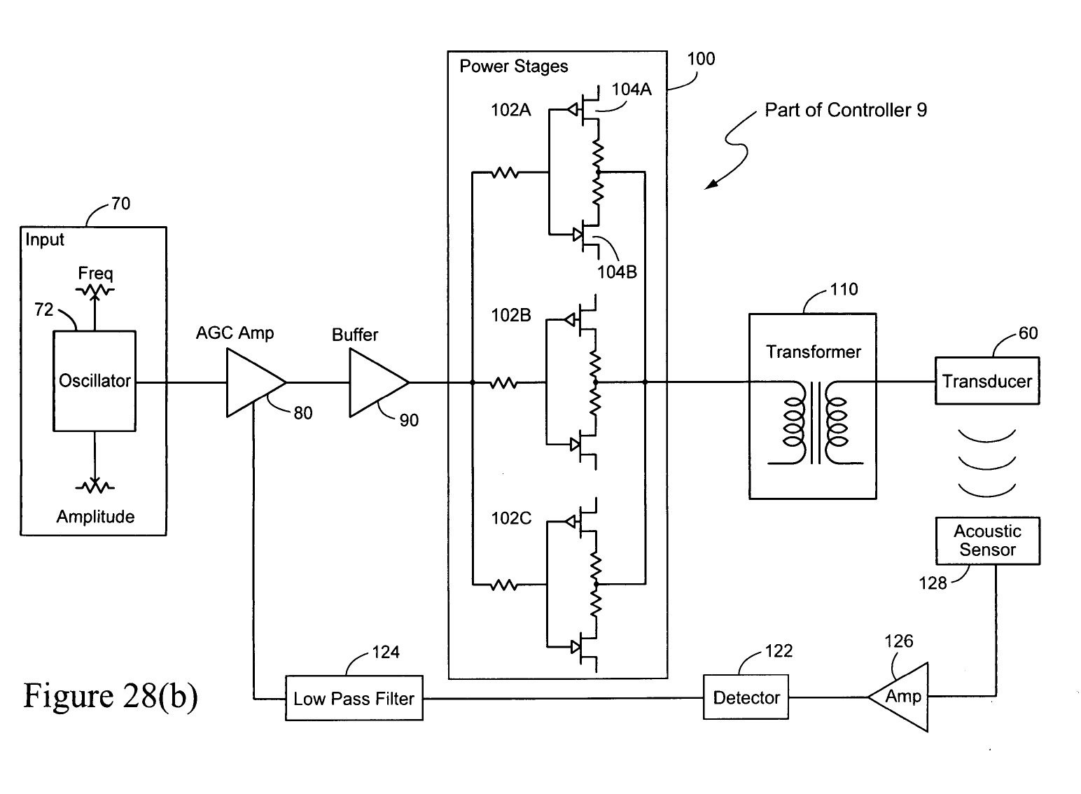

FIG. 28(a) is non-limiting schematic diagram of an example relatively high frequency and high current oscillator/amplifier for driving a large-area ultrasonic transducer. The relatively high frequency range of operation preferably corresponds to the frequency range of the large area transducer. One non-limiting example frequency range is from about 100 KHz-10 MHz. The input stage 70 includes an oscillator 72. An automatic gain controlled (AGC) amplifier stage 80 receives the signal from the input stage 70 and amplifies it, e.g., an example gain is five. The amplified signal is buffered in a high frequency, high current monolithic buffer 90 which drives an output power stage 100 including three pairs 102A-102C of output FET's 104A, 104B in a push-pull configuration. The high-frequency, high-current buffer, combined with the three pairs of FET's configured in parallel allows the drive circuit to achieve higher switching speeds and current capacity needed to drive a large area ultrasonic transducer 60 at high power at frequencies exceeding 1 MHz. Matching the low impedance matching of the large area transducer 60 means that the amplifier must be able to drive the transducer 60 at a high current level. Ohm's law dictates that for a constant voltage, a low impedance results in a high current. A typical large area ultrasonic transducer may have an impedance on the order of several ohms, e.g., 2-4 ohms.

The output FET's preferably have low drain to source resistance when the device is in full conduction, low gate capacitance, and high drain current specifications. The three output FET pairs 102A-102C are connected in parallel to increase the available output current to the load as well as lower the output impedance of the amplifier. The low output impedance allows operation up to several MHz by keeping the time constants between the output FET pairs 102A-102C and the reactive transducer load very short.

The output stage 100 is followed by a transmission line transformer 110 and a low impedance cable output. The transmission line transformer 110 in a non-limiting example test device is a 4:2 impedance matching design that allows the amplifier to drive the transducer load with only a moderate number of output devices, reducing the size, cost, and cooling requirements of the amplifier. The low impedance amplifier circuit in FIG. 28(a) does this by essentially doubling the impedance of the transducer from the amplifier's perspective. This halves the amount of current the amplifier must supply at 2.2 MHz, for example, and drops the power dissipation in the FET's by a factor of 4 in this non-limiting example. In order to reduce the size of the amplifier, the transmission line transformer 110 can be co-located with the transducers, which will simplify the cabling between the amplifier and the transducer.

There are additional non-limiting design features that improve the operation of the amplifier circuitry used to drive a large area ultrasonic transducer. One is the production of a square wave output waveform with no ringing, which results in maximum energy transfer to the transducer. Another is minimal feedback to allow a high slew rate and taking advantage of device capacitance to suppress ringing. Still further, the frequency and amplitude of the large area ultrasonic transducer drive signal are preferably adjusted so that the oscillator/amplifier is tuned to the individual transducer it is driving. This allows the acoustic outputs of the transducer or transducers in the debubbling apparatus to be matched. Impedance matching between the oscillator/amplifier and its corresponding transducer may be handled by the transmission line transformer 110 driving a low impedance output cable assembly.

FIGS. 28(a) and 28(b) also show an automatic gain control (AGC) system which may be desirable. In FIG. 28(a), a current detector 122 may receive the transformer output via a resistor ladder to monitor the power dissipated in the transducer with a feedback via a low pass filter 124 to a control input of the AGC amplifier stage 80 so that the acoustic output may be maintained at a consistently safe level. The current sensor 122 monitors the amount of current supplied to the transducer 60 which changes as the transducer heats up. The rising temperature decreases the transducer impedance. The feedback to the AGC stage 80 controls the drive level of the amplifier for the transducer 60, and ultimately, controls the acoustic output of the transducer 60. The AGC feedback and amplifier stage 80 also take into account the acoustic impedance of the medium into which the transducer 60 is sending ultrasonic waves.

In another non-limiting example shown in FIG. 28(b), the acoustic output of the transducer 60 is directly measured within the debubbling apparatus using a second ultrasound transducer 128 mounted within the debubbling apparatus. Because plastics provide good acoustic coupling between a ceramic transducer and water, a polyvinylidene fluoride (PVDF) or other polymer transducer is an example of such a second transducer 128 that could either be applied to the front surface of the drive transducer 60 or on the acoustic window 17. This second acoustic transducer 128 converts the ultrasonic output into a voltage signal which is amplified in amplifier 126, detected in detector 122, filtered in low pass filter 124 and used to feed the control of the AGC stage 80.

Both examples of FIGS. 28(a) and 28(b) provide a desirable control function that prevents an “open loop” feedback situation wherein increasing current is supplied to a failing transducer, causing electrical heating that destroys the amplifier circuit, the transducer, or both.

In general, the large area transducer implementation of the second embodiment reduces the number of chambers, thus lowering the prime volume of the debubbling apparatus, which means that the debubbling apparatus can be used in a bypass circuit with less hemodilution.

With respect to all of the example embodiments, by using ultrasonic radiation force in conjunction with mechanical features for debubbling a fluid (e.g., swirling flow, porous mesh filters, etc.), the technology described above removes bubbles from fluids more effectively than devices that just use ultrasound or mechanical features. In addition, the technology may be integrated into current CPB components and does not add fluid volume to the bypass circuit. In fact, by more effectively removing bubbles from fluid, it is possible to reduce the fluid volume of CPB circuit components, which results in less use of transfused blood during CPB surgery to maintain hematocrit and a reduced risk of systemic inflammation. Use of ultrasonic radiation force may also serve to reducing the total amount of mechanical filters within the CPB circuit; this may have the beneficial effect of reducing damage to red blood cells caused when the red blood cells hit the mesh fibers within these filters.

Although various example embodiments have been shown and described in detail, the claims are not limited to any particular embodiment or example. None of the above description should be read as implying that any particular element, step, range, or function is essential such that it must be included in the claims scope. Reference to an element in the singular is not intended to mean “one and only one” unless explicitly so stated, but rather “one or more.” The scope of patented subject matter is defined only by the claims. The extent of legal protection is defined by the words recited in the allowed claims and their equivalents. All structural and functional equivalents to the elements of the above-described example embodiment that are known to those of ordinary skill in the art are expressly incorporated herein by reference and are intended to be encompassed by the present claims. Moreover, it is not necessary for a device or method to address each and every problem sought to be solved by the present invention for it to be encompassed by the present claims. No claim is intended to invoke paragraph 6 of 35 USC §112 unless the words “means for” or “step for” are used. Furthermore, no feature, component, or step in the present disclosure is intended to be dedicated to the public regardless of whether the feature, component, or step is explicitly recited in the claims.

1. A vessel for removing bubbles from a fluid comprising:
a fluid inlet port for receiving the fluid;
an air outlet for removing air in the fluid from the vessel;
one or more ultrasonic transducers arranged to transmit one or more ultrasonic beams through the received fluid in a first direction to move bubbles in the fluid towards the air outlet port;
a fluid outlet port for outputting the fluid insonified by the one or more ultrasonic beams;
a conduit structure for conveying the one or more ultrasonic beams through the vessel in a first direction towards the air outlet; and
an interface that prevents reflection of one or more ultrasonic beams in a direction generally opposite the first direction.

2. The vessel in claim 1, wherein the interface is an air/fluid interface that reduces acoustic energy that may be reflected in the vessel back toward the fluid outlet port.

3. The vessel in claim 2, wherein the air/fluid interface is curved or arced when the one or more ultrasound transducer(s) are operational.

4. The vessel in claim 3, wherein a force of the transmitted ultrasonic beams produce an acoustic streaming effect that dissipates the energy of the ultrasonic beams in the first direction and minimizes reflected ultrasonic beams that are reflected back in the opposite direction, and wherein reflected ultrasonic beams reflected back in the opposite direction reduce the radiation force on bubbles in the first direction.

5. The vessel in claim 1, wherein the interface includes an ultrasonic reflector mounted in the vessel for reflecting the one or more ultrasonic beams away from the fluid outlet port to reduce or prevent reflection of the ultrasonic beam off an interior surface in the vessel directed towards the blood outlet port.

6. The vessel in claim 1, wherein a shape of an interior portion of the vessel provides the interface.

7. The vessel in claim 6, wherein the interior portion includes a fluid inlet chamber coupled to the fluid inlet port.

8. The vessel in claim 1, wherein a cross section of each conduit in the conduit structure substantially matches a cross section of its conveyed ultrasonic beam.

9. The vessel in claim 1, further comprising:
an acoustically transparent material separating the one or more ultrasonic transducers from the fluid inlet port and the fluid outlet port.

10. The vessel in claim 9, wherein the acoustically transparent material is shaped to adjust the ultrasound beam so that a profile of the ultrasound beam approximates the dimensions of the opening in the barrier.

11. The vessel in claim 1, further comprising:
means for removing heat from vessel caused by the ultrasonic transducer.

12. The vessel in claim 1, wherein the one or more ultrasonic transducers includes multiple ultrasonic transducers, wherein the conduit structure includes multiple connecting tubes for conveying the ultrasonic beams from the multiple ultrasonic transducers through the vessel in the first direction.

13. The vessel in claim 12, further comprising an ultrasonic standoff region between the multiple transducers and the connecting tubes, wherein a length of the ultrasound standoff region is such that the width of each ultrasound beam as it enters a corresponding connecting tube substantially matches a width of that connecting tube.

14. The vessel in claim 1, wherein the one or more ultrasonic transducers includes one ultrasonic transducer comprised of a tiled transducer array which at least reduce vibrations that can cause an unified transducer to fail.

15. The vessel in claim 1, wherein the one or more ultrasonic transducers includes one large area ultrasonic transducer driven by an amplifier whose frequency response and impedance substantially match those of the one large area ultrasonic transducer.

16. The vessel in claim 15, wherein the frequency range of the one large area ultrasonic transducer is 1 MHz or more.

17. The vessel in claim 16, wherein the impedance of the one large area ultrasonic transducer is on the order of several ohms.

18. The vessel in claim 15, wherein the amplifier includes an automatic gain control that adjusts an output power to the transducer if the transducer impedance changes due to heating or other external influences.

19. A system for removing gaseous emboli from blood, comprising:
a blood circuit receiving blood from a patient;
a pump coupled to the blood circuit for pumping the blood through the blood circuit;
a vessel coupled to the blood circuit for removing gaseous emboli from blood including:
a blood inlet port for receiving the blood;
an emboli outlet for removing gaseous emboli in the blood from the vessel;
one or more ultrasonic transducers mounted in the vessel and arranged to transmit one or more ultrasonic beams through the received blood to move gaseous emboli in the blood towards the gaseous emboli outlet;
a blood outlet port for outputting the blood insonified by the one or more ultrasonic beams; and
a conduit structure for conveying the one or more ultrasonic beams through the vessel in a first direction towards the air outlet; and
an interface that prevents reflection of one or more ultrasonic beams in a direction generally opposite the first direction.

20. The system in claim 19, wherein the interface is an air/blood interface that reduces acoustic energy that may be reflected in the vessel back toward the blood outlet port.

21. The system in claim 20, wherein a force of the transmitted ultrasonic beams produce an acoustic streaming effect that dissipates the energy of the ultrasonic beams in the first direction and minimizes reflected ultrasonic beams that are reflected back in the opposite direction, and wherein reflected ultrasonic beams reflected back in the opposite direction reduce the radiation force on gaseous emboli in the first direction.

22. The system in claim 19, wherein the interface includes an ultrasonic reflector mounted near the gaseous emboli outlet for reflecting the one or more ultrasonic beams away from the blood outlet port to reduce or prevent reflection of the one or more ultrasonic beams off an interior surface in the vessel directed towards the blood outlet port; and
a controller for controlling the one or more ultrasonic transducers and the pump.

23. The system in claim 19, wherein a shape of an interior portion of the vessel provides the interface.

24. The system in claim 23, wherein the interior portion includes a blood inlet chamber coupled to the blood inlet port.

25. The system in claim 19, wherein a cross section of each conduit in the conduit structure substantially matches a cross section of its conveyed ultrasonic beam.

26. The system in claim 19, further comprising:
an acoustically transparent material separating the one or more ultrasonic transducers from the blood inlet port and the blood outlet port.

27. The system in claim 26, wherein the acoustically transparent material is shaped to adjust the ultrasound beam so that a profile of the ultrasound beam approximates the dimensions of the opening in the barrier.

28. The system in claim 19, wherein the one or more ultrasonic transducers includes multiple ultrasonic transducers, wherein the conduit structure includes multiple connecting tubes for conveying the ultrasonic beams from the multiple ultrasonic transducers through the vessel in the first direction.

29. The system in claim 27, further comprising an ultrasonic standoff region between the multiple transducers and the connecting tubes, wherein a length of the ultrasound standoff region is such that the width of each ultrasound beam as it enters a corresponding connecting tube substantially matches a width of that connecting tube.

30. The system in claim 19, wherein the one or more ultrasonic transducers includes one ultrasonic transducer comprised of a tiled transducer array which at least reduce vibrations that can cause an untiled transducer to fail.

31. The system in claim 19, wherein the one or more ultrasonic transducers includes one large area ultrasonic transducer driven by an amplifier whose frequency response and impedance substantially match those of the one large area ultrasonic transducer.

32. A method for debubbling a liquid comprising:
introducing the liquid to a vessel through a fluid inlet;
causing the liquid to flow through the vessel toward a first outlet;
operating one or more ultrasonic transducers to transmit one or more ultrasonic beams through a conduit structure of the vessel and toward an air outlet;
withdrawing a stream of insonified liquid through the first outlet;
withdrawing a stream of liquid containing entrained air bubbles through the air outlet or allowing release of air bubbles from the fluid into the air at fluid/air interface in the vessel; and
using an interface to prevent reflection of one or more ultrasonic beams in a direction generally opposite the first direction.

33. The method in claim 32, further comprising:
shaping the ultrasound beam so that a profile of the ultrasound beam approximates the dimensions of the opening in the barrier.

34. The method in claim 32, further comprising:
using an oscillator and an amplifier to generate a high-current, high-frequency drive signal that matches the impedance of the one or more ultrasound transducers, and
driving the one or more transducers using the generated drive signal.

35. The method in claim 32, wherein the interface is an air/fluid interface that reduces acoustic energy that may be reflected in the vessel back toward the fluid outlet port.

36. The method in claim 32, wherein a force of the transmitted ultrasonic beams produce an acoustic streaming effect that dissipates the energy of the ultrasonic beams in the first direction and minimizes reflected ultrasonic beams that are reflected back in the opposite direction, and wherein reflected ultrasonic beams reflected back in the opposite direction reduce the radiation force on gaseous emboli in the first direction.

37. The method in claim 32, further comprising using an ultrasonic reflector mounted near the gaseous emboli outlet as the interface to reflect the one or more ultrasonic beams away from the fluid outlet port to reduce or prevent reflection of the one or more ultrasonic beams off an interior surface in the vessel directed towards the fluid outlet.

38. The method in claim 32, wherein a shape of an interior portion of the vessel provides the interface.
